(19) United States
(12) Patent Application Publication
(10) Pub. No.: US 2025/0288495 A1
(43) Pub. Date: Sep. 18, 2025

(54) Dispensing device and user terminal for managing cartridge module

(71) Applicant: ALGOCARE CORPORATION, Seoul (KR)

(72) Inventors: Jiwon JOUNG, Seoul (KR); Pil Lip YOUN, Seoul (KR)

(21) Appl. No.: 18/833,482

(22) PCT Filed: Nov. 9, 2022
(86) PCT No.: PCT/KR2022/017566

§ 371 Date: Feb. 10, 2025

(30) Foreign Application Priority Data
Feb. 9, 2022 (KR) 10-2022-0016894
Jul. 7, 2022 (KR) 10-2022-0083482

(51) Int. Cl. A61J 7/00

(52) U.S. Cl. CPC A61J 7/0084

(57) ABSTRACT

The present invention is directed to a dispensing device and user terminal for managing a cartridge module. A dispensing device according to an example includes: an accommodation unit configured to provide an internal space for accommodating a plurality of cartridge modules; a discharge unit configured to discharge tablets from each of the plurality of cartridge modules; detection sensors provided to corresponding to the plurality of cartridge module, respectively, and each configured to count the number of tablets discharged from each of the plurality of cartridge modules; and a control unit electrically connected to the electrodes of the accommodation unit, the discharge unit, and the detection sensors, and configured to control the discharge of the tablets. The control unit performs control to discharge the tablets in response to a discharge request signal, and also performs control to update residual quantity information.

### TECHNICAL FIELD

The present invention relates to a dispensing device and user terminal for managing a cartridge module.

### BACKGROUND ART

As the nutrient market grows and users' needs are diversified, personalized nutrient services that analyze the health states of users and combine and provide appropriate nutrients accordingly are becoming more active. Such personalized nutrient services are provided in such a manner as to collect the health information of users through questionnaires and/or the like, derive nutritional ingredients deemed necessary by analyzing the collected health information of users, and combine and provide nutrients that satisfy the corresponding nutritional ingredients.

### DISCLOSURE

### Technical Problem

An object to be achieved in the present application is to provide a user with information about the residual quantity of nutrients contained in a cartridge module that is mounted in a dispensing device.

Objects to be achieved in the present application are not limited to the object described above, and objects not described above can be clearly understood by those having ordinary skill in the art, to which the present application pertains, from the present application.

### Technical Solution

According to an example of the present invention, there is provided a dispensing device, including: an accommodation unit configured to provide an internal space for accommodating a plurality of cartridge modules; a discharge unit configured to discharge tablets from each of the plurality of cartridge modules; detection sensors provided to corresponding to the plurality of cartridge module, respectively, and each configured to count the number of tablets discharged from each of the plurality of cartridge modules; and a control unit electrically connected to the electrodes of the accommodation unit, the discharge unit, and the detection sensors, and configured to control the discharge of the tablets; wherein the control unit performs control to discharge the tablets from at least one of the plurality of cartridge modules through the discharge unit in response to a discharge request signal for the tablets, and, after the tablets have been discharged from the at least one cartridge module, performs control to update residual quantity information for the at least one cartridge module based on discharged quantity information about the number of tablets detected by the detection sensor.

In this case, each of the plurality of cartridge modules may include a cartridge identification unit configured to store information about the cartridge module, the accommodation unit may have cartridge module interfaces each electrically connected to the cartridge identification unit, and the control unit may perform control to obtain information about the cartridge module from the cartridge identification unit through the cartridge module interface when each of the plurality of cartridge modules is mounted in the accommodation unit.

As an example, the cartridge identification unit may be provided in the form of a read/write-enabled memory chip, the information about the cartridge module may include the residual quantity information, and the control unit may perform control to obtain the residual quantity information from the memory chip when each of the plurality of cartridge modules is mounted in the accommodation unit, and, after the residual quantity information has been updated, performs control to store the residual quantity information in the memory chip.

Furthermore, the dispensing device may further include an output unit configured to display information about the plurality of cartridge modules, and the output unit may display a plurality of images for the plurality of cartridge modules to correspond to the arrangement of the plurality of cartridge modules in the accommodation unit, and display information about tablets accommodated in each of the plurality of cartridge modules and residual quantity information together in each of the plurality of images.

In this case, the output unit may further display insufficient quantity information for an image representative of a cartridge module whose residual quantity information is smaller than a reference value among the plurality of images.

Furthermore, the accommodation unit may include a top cover configured to cover the inner space of the accommodation unit, and a display unit including a plurality of indication lamps located in respective portions corresponding to the plurality of cartridge modules in the internal space of the accommodation unit, and the control unit may, when the accommodation unit is opened, perform control to blink a target indication lamp corresponding to a cartridge module whose residual quantity is smaller than a predetermined reference value among the plurality of indicator lamps, and, when the accommodation unit is closed or the cartridge module whose residual quantity is smaller than the predetermined reference value is separated from the accommodation unit, perform control to stop blinking the target indication lamp.

Furthermore, the control unit may, when a new cartridge module is mounted after the cartridge module whose residual quantity is smaller than the predetermined reference value has been separated, control the target indication lamp by using at least one of the identification information and residual quantity information of the new cartridge module.

In this case, the control unit may perform control to obtain the at least one of the identification information and residual quantity information of the new cartridge module from a memory chip included in the new cartridge module.

Furthermore, the control unit may, when the cartridge module whose residual quantity is smaller than the predetermined reference value is separated and then a cartridge module whose residual quantity is smaller than the predetermined reference value is mounted, blink again the target indication lamp whose blinking was stopped.

According to an example of the present invention, there is provided a user terminal, including: a terminal storage unit including memory; a terminal control unit including a processor electrically connected to the terminal storage unit, and configured to execute commands stored in the terminal storage unit; a terminal communication unit configured to transmit and receive information to and from a server or dispensing device under the control of the terminal control unit; and a terminal output unit configured to output results according to operation of the terminal control unit; wherein the terminal control unit performs control to receive residual quantity information about a plurality of cartridge modules mounted in the dispensing device from the server or dispensing device and to output the residual quantity information through the terminal output unit, and, after one or more tablets have been discharged from the plurality of cartridge modules, performs control to update and output the residual quantity information.

In this case, the terminal output unit may display the residual quantity information together with information about tablets accommodated in each of the plurality of cartridge modules.

Furthermore, the terminal output unit may display a plurality of images for the plurality of cartridge modules to correspond to the arrangement of the plurality of cartridge modules in the dispensing device.

The terminal control unit may, when the residual quantity information for at least one of the plurality of cartridge modules is smaller than a preset reference value, perform control to receive insufficient quantity information for the at least one module from the server or dispensing device; and the terminal output unit may output the insufficient quantity information for the at least one cartridge module together with the residual quantity information.

Furthermore, the terminal output unit may display, among the plurality of images representative of the plurality of cartridge modules, an image representative of a cartridge module whose residual quantity information is smaller than a predetermined reference value so that the image can be distinguished from an image representative of a cartridge module whose residual quantity information is equal to or larger than the reference value.

The technical solutions of the present application are not limited to the solutions described above, and solutions not described above can be clearly understood by those having ordinary skill in the art, to which the present application pertains, from the present application.

### Advantageous Effects

According to an embodiment, information about the residual quantity of nutrients contained in a cartridge module that is mounted in a dispensing device may be provided to a user.

Effects of the invention of the present application are not limited to the effect described above, and effects not described above can be clearly understood by those having ordinary skill in the art, to which the present application pertains, from the present application.

### DESCRIPTION OF DRAWINGS

FIG. 1 is a diagram of a system according to an embodiment;

FIG. 2 is a block diagram of a server according to an embodiment;

FIG. 3 is a block diagram of a user terminal according to an embodiment;

FIGS. 4 and 5 are diagrams of an example of a cartridge module according to an embodiment;

FIGS. 6 to 8 are diagrams of an example of a dispensing device according to an embodiment;

FIG. 9 is a block diagram of the example of the dispensing device according to the embodiment;

FIG. 10 is a diagram of an example of a nutrient detection sensor according to an embodiment;

FIG. 11 is a flowchart of an example of a method of managing cartridge modules according to an embodiment;

FIGS. 12a to 12d are diagrams of examples of displaying cartridge module management information, which is an example of outputting cartridge module management information according to an embodiment;

FIG. 13 is a diagram of another example of displaying cartridge module management information, which is an example of outputting cartridge module management information according to an embodiment;

FIGS. 14 and 15 are flowcharts of a first embodiment in which a server, a user terminal, and a dispensing device perform a residual quantity management method while operating in conjunction with each other,

FIGS. 16 and 17 are flowcharts of a second embodiment in which a server, a user terminal, and a dispensing device perform a residual quantity management method while operating in conjunction with each other,

FIGS. 18 and 19 are flowcharts of a third embodiment in which a server, a user terminal, and a dispensing device perform a residual quantity management method while operating in conjunction with each other,

FIGS. 20 and 21 are flowcharts of a fourth embodiment in which a server, a user terminal, and a dispensing device perform a residual quantity management method while operating in conjunction with each other,

FIG. 22 is a flowchart of another example of a method of managing a cartridge module according to an embodiment;

FIG. 23 is a diagram of an example in which a dispensing device according to an embodiment controls a display unit; and

FIG. 24 is a flowchart of a more specific illustration of another example of the method of managing a cartridge module shown in FIG. 22.

### BEST MODE

The embodiments described in the present application are intended to clearly describe the spirit of the present application to those having ordinary skill in the art to which the present application pertains and, thus, are not limited by the embodiments described in the present application. The scope of the present application should be construed to include modifications or variations that do not depart from the spirit of the present application.

General terms that are currently widely used are selected as the terms used in the present application by taking into consideration their functions in the present application as much as possible. These may vary depending on the intentions of those having ordinary skill in the art to which the present application pertains, customs, or the emergence of new technologies. However, when a specific term is defined and used to have a desired meaning, the meaning of the term will be described separately. Therefore, the terms used in the present application should be interpreted not based on only the names of the terms but based on the actual meanings of the terms and the overall context of the present application.

The drawings of the present application are intended to easily illustrate the present application. The shapes shown in the drawings may be exaggerated as necessary to help the understanding of the present application, so that the present application is not limited by the drawings.

In the present application, when it is determined that a detailed description of a known configuration or function related to the present application may obscure the gist of the present application, the detailed description thereof will be omitted as necessary. Furthermore, numbers (e.g., first, second, etc.) used in the description of the present application are merely identification symbols intended to distinguish one component from other components, unless otherwise specified.

A singular expression includes plural expressions unless the context clearly dictates otherwise. In the present application, it should be understood that the terms such as “comprise” or “include” are intended to designate the presence of features, numbers, steps, operations, components, parts, or combinations thereof described in the specification, but do not exclude in advance the probability of the presence or addition of one or more other features, numbers, steps, operations, components, parts, or combinations thereof.

The present specification is directed to a dispensing device that provides nutrients to a user. The dispensing device is mounted with a cartridge module containing nutrients, and the nutrients contained in the cartridge module are discharged and provided to a user by the nutrient discharge operation of the dispensing device. In this case, when all the nutrients contained in the cartridge module run out or become insufficient, the user replaces the used cartridge module with a new cartridge module and continues to use the dispensing device.

In the present specification, the initially charged quantity information may be information about the quantity of nutrients with which a cartridge module is initially charged. As an example, the initial filling amount information may be information about the number of nutrients with which a cartridge module is initially charged. As another example, the initially charged quantity information may be information about the weight of nutrients with which a cartridge module is initially charged.

In the present specification, the discharged quantity information may be information about the quantity of nutrients that is discharged from a cartridge module. As an example, the discharged quantity information may be information about the number of nutrients that are discharged from a cartridge module. As another example, the discharged quantity information may be information about the weight of nutrients that are discharged from a cartridge module.

In the present specification, the residual quantity information may be information about the residual quantity of nutrients that are contained in a cartridge module. As an example, the residual quantity information may be information about the number of nutrients that are contained in a cartridge module. As another example, the residual quantity information may be information about the weight of nutrients that are contained in a cartridge module. As still another example, the residual quantity information may be information about the ratio of the residual quantity of nutrients to the initially charged quantity of nutrients.

Hereinafter, a method, server, user terminal, dispensing device, and system for managing a cartridge module according to the present application will be disclosed.

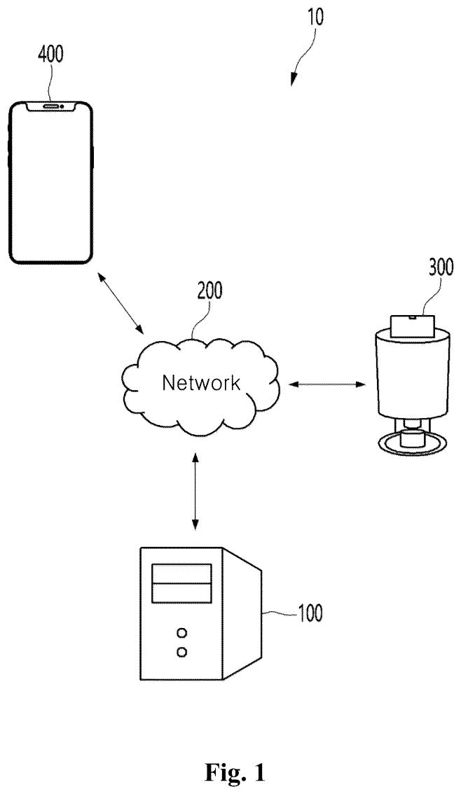

FIG. 1 is a diagram of a system 10 according to an embodiment.

Referring to FIG. 1, a network 200 may communicatively connect any two or more of, e.g., a server 100, a dispensing device 300, and a user terminal 400 to each other (directly or indirectly).

The communication method of the network 200 is not limited, and may include not only communication methods utilizing communication networks (e.g., a mobile communication network, the wired Internet, the wireless Internet, and a broadcasting network) that the network 200 may include, but also short-distance wireless communication.

As an example, the network 200 may be implemented as any type of wired/wireless network, such as a local area network (LAN), a wide area network (WAN), a value added network (VAN), a personal area network (PAN), a mobile radio communication network, or a satellite communication network.

The dispensing device 300 may discharge nutrients.

The dispensing device 300 may be mounted with one or more cartridge modules. The dispensing device 300 may be mounted with a plurality of cartridge modules.

One cartridge module may contain one type of nutrients. As an example, a magnesium cartridge module may contain magnesium nutrients. As another example, a multi-mineral cartridge module may contain multi-mineral nutrients.

The nutrients may be provided in any one of various forms, such as a solid form (e.g., tablets, pills, granules, etc.), a capsule form, a powder form, and a jelly form.

The dispensing device 300 may discharge nutrients contained in a mounted cartridge module.

The dispensing device 300 may discharge one or more nutrients based on a nutrient discharge request signal. As an example, the dispensing device 300 may receive a nutrient discharge request signal from the server 100 and discharge one or more nutrients in response to the received nutrient discharge request signal. The nutrient discharge request signal may illustratively include information about the type of nutrients to be discharged, the number of nutrients to be discharged, or a combination thereof, but is not limited thereto.

The server 100 may be implemented as a computer device that communicates with the user terminal 400 and the dispensing device 300 over the network 200 and provides commands, codes, files, content, and services.

The server 100 may manage residual quantity information, which is information about the residual quantity of nutrients. As an example, the server 100 may receive residual quantity information from the dispensing device 300 and store the received residual quantity information.

The user terminal 400 may be a fixed terminal implemented as a computer device, or a mobile terminal. As an example, the user terminal 400 may be one of a smartphone, a mobile phone, a tablet personal computer (PC), a computer, a notebook computer, and a personal digital assistant (PDA).

The user terminal 400 may be used by a user. The user may be the user of the user terminal 400, but may also refer to the user of an account registered for a service provided by the server 100. The server 100 transmitting or providing specific information to a user may mean providing specific information to the user by transmitting the information to the user terminal 400 of an account registered with the server 100.

The user terminal 400 may be provided with an application intended to perform the above-described functions, and may be operated in conjunction with the server 100 through the application.

The configuration of the system will be described in more detail below.

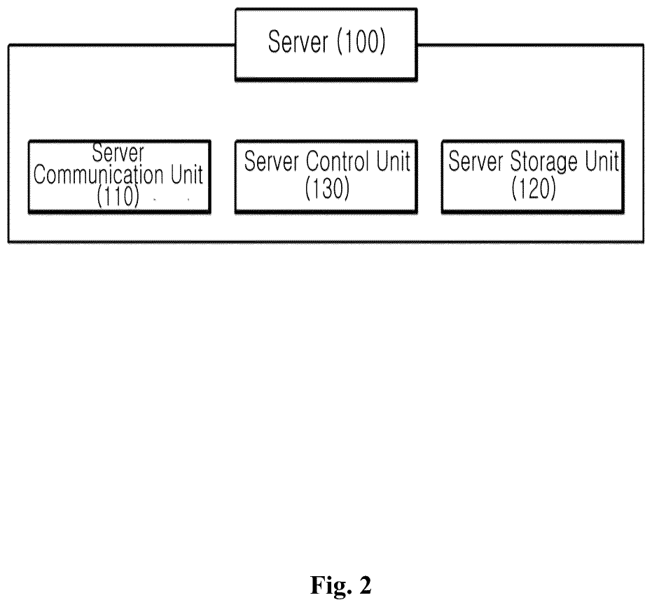

FIG. 2 is a block diagram of the server 100 according to an embodiment. Referring to FIG. 2, the server 100 may include a server communication unit 110, a server storage unit 120, and a server control unit 130.

The server 100 may communicate with the outside through the server communication unit 110. As an example, the server 100 may receive initially charged quantity information, residual quantity information, or discharged quantity information from the dispensing device 300 through the server communication unit 110. As another example, the server 100 may transmit residual quantity information to the user terminal 400 through the server communication unit 110. As still another example, the server 100 may transmit a nutrient discharge request signal to the dispensing device 300 through the server communication unit 100.

The server communication unit 110 may perform wired or wireless communication. The server communication unit 110 may be, e.g., a wired/wireless Local Area Network (LAN) module, a WAN module, an Ethernet module, a Bluetooth module, a Zigbee module, a Universal Serial Bus (USB) module, an IEEE 1394 module, a Wi-Fi module, a mobile communication module, a satellite communication module, or a combination thereof, but is not limited thereto.

The server 100 may store various types of data, programs, or applications necessary for operation in the server storage unit 120. Each of the programs or applications stored in the server storage unit 120 may include one or more instructions. Each of the programs or applications stored in the server storage unit 120 may be executed by the server control unit 130.

The server storage unit 120 may store the information obtained by the server 100. The server storage unit 120 may store the results of operations performed by the server 100, etc.

The server storage unit 120 may be the internal storage of the server 100. Alternatively, the server storage unit 120 may be external storage such as cloud storage.

The server storage unit 120 may be, e.g., a hard disk, flash memory, a solid-state drive (SSD), random access memory (RAM), read-only memory (ROM), electrically erasable programmable read-only memory (EEPROM), magnetic memory, a magnetic disk, an optical disk, or a combination thereof, but is not limited thereto.

The server 100 may perform the processing and calculation of various types of information through the server control unit 130. The server control unit 130 may control other components constituting the server 100.

The server control unit 130 may be implemented as a computer or similar device depending on hardware, software, or a combination thereof. In terms of hardware, the server control unit 130 may be one or more processors. Alternatively, the server control unit 130 may be provided as processors that are physically spaced apart from each other and collaborate through communication. The server control unit 130 may be, e.g., a central processing unit (CPU), a graphics processing unit (GPU), a digital signal processor (DSP), a state machine, an application-specific integrated circuit (ASIC), a radio-frequency integrated circuit (RFIC), or a combination thereof, but is not limited thereto. The server control unit 130 may be provided in the form of a program that drives a hardware server control unit 130 in terms of software.

Unless otherwise specified below, the operation of the server 100 may be interpreted as performed by or under the control of the server control unit 130.

Not all of the components shown in FIG. 2 are essential components of the server 100, and at least some of the components of the server 100 shown in FIG. 2 may be omitted. Furthermore, the server 100 may additionally include one or more components that are not shown in FIG. 2. For example, the server 100 may further include a server output unit including a display, a speaker, or a combination thereof.

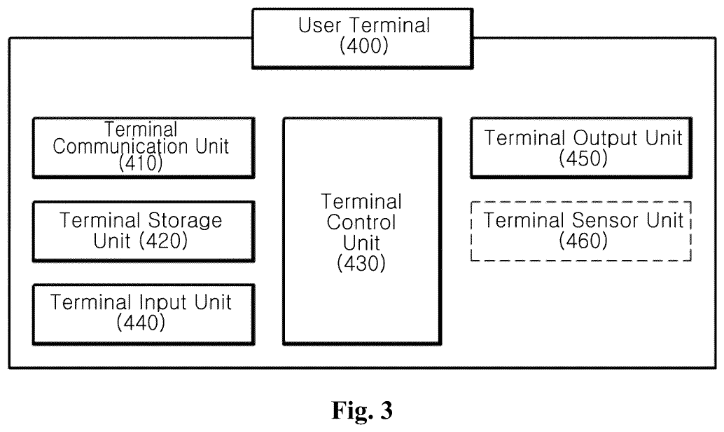

FIG. 3 is a block diagram of the user terminal 400 according to an embodiment. Referring to FIG. 3, the user terminal 400 may include a terminal communication unit 410, a terminal storage unit 420, a terminal control unit 430, a terminal input unit 440, and a terminal output unit 450.

Like the server communication unit 110 described above, the user terminal 400 may communicate with the outside through the terminal communication unit 410. As an example, the user terminal 400 may receive residual quantity information from the server 100 or dispensing device 300 through the terminal communication unit 410. Redundant descriptions of the parts of the terminal communication unit 410 that are the same as or similar to those of the server communication unit 110 will be omitted.

Like the server storage unit 120 described above, the user terminal 400 may store various types of data, programs, or applications necessary for operation in the terminal storage unit 420. The terminal storage unit 420 may store information obtained by the user terminal 400. Redundant descriptions of the parts of the terminal storage unit 420 that are the same as or similar to those of the server storage unit 120 will be omitted.

Like the server control unit 130 described above, the user terminal 400 may perform the processing and calculation of various types of information through the terminal control unit 430. Redundant descriptions of the parts of the terminal control unit 430 that are the same as or similar to those of the server control unit 130 will be omitted.

The user terminal 400 may obtain user input through the terminal input unit 440. The user input may be, e.g., key input, touch input, voice input, or a combination thereof, but is not limited thereto.

The terminal input unit 440 may be implemented as a commonly used user input device. The user input device may be, e.g., a touch sensor configured to detect touches of a user, a microphone configured to receive voice signals, a gesture sensor configured to recognize gestures of a user, a keyboard, a mouse, or a combination thereof, but is not limited thereto.

The user terminal 400 may output information through the terminal output unit 450. As an example, the user terminal 400 may output residual quantity information through the terminal output unit 450. The output of information may be visual, auditory, and/or tactile output, but is not limited thereto. The output of information may be performed in various forms.

The terminal output unit 450 may be implemented as a display configured to output images, a speaker configured to output sounds, a haptic device configured to generate vibrations, or various other types of output means.

Alternatively, the terminal output unit 450 may also be implemented in the form of an output interface (a USB port, a PS/2 port, or the like) that connects an external output device configured to output information to the user terminal 400, instead of a device that outputs information to the outside on its own.

The terminal output unit 450 may be integrated with the terminal input unit 440. For example, when the terminal output unit 450 is a display, the terminal output unit 450 may be in the form of a touch display that is integrated with a touch sensor, which is the terminal input unit 440.

Referring to FIG. 3, the user terminal 400 may further include a terminal sensor unit 460. The terminal sensor unit 460 may detect information related to a user's health, and may include, e.g., an acceleration sensor, a location sensor, or a combination thereof. As an example, the user terminal 400 may obtain a user's quantity-of-motion information through the terminal sensor unit 460.

Not all of the components shown in FIG. 3 are essential components of the user terminal 400, and at least some of the components of the user terminal 400 shown in FIG. 3 may be omitted. Furthermore, the user terminal 400 may additionally include one or more components not shown in FIG. 3.

Prior to the description of the dispensing device in more detail, a cartridge module that can be accommodated or mounted in the dispensing device will first be described.

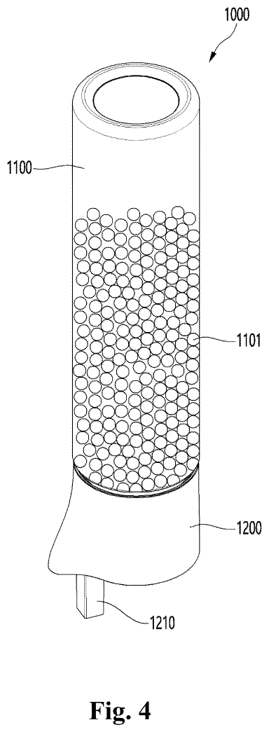

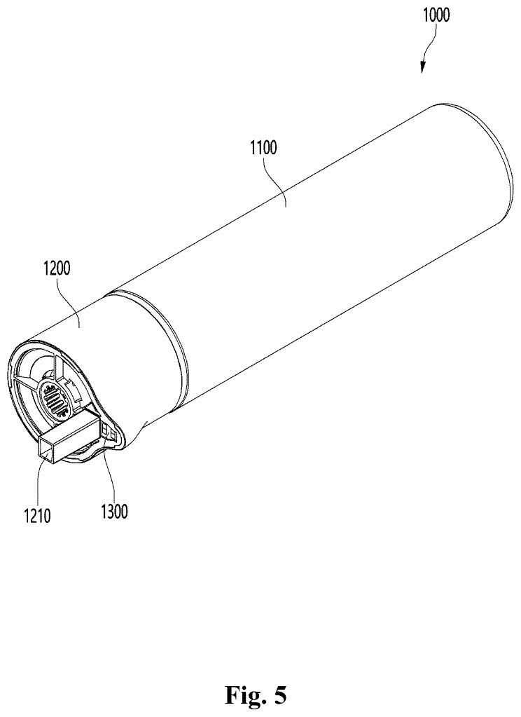

FIGS. 4 and 5 are diagrams of an example of a cartridge module 1000 according to an embodiment. The example of the cartridge module 1000 will be described with reference to FIGS. 4 and 5 below.

The cartridge module 1000 is accommodated or mounted in the dispensing device, and nutrients 1101 contained in the cartridge module 1000 may be discharged by the nutrient discharge operation of the dispensing device.

The cartridge module 1000 may include a nutrient storage part 1100, and a cartridge head 1200.

The nutrient storage part 1100 and the cartridge head 1200 may be selectively coupled to and separated from each other. For example, screw threads are formed on the outside of one area of the nutrient storage part 1100 and screw threads are formed on the inside of one area of the cartridge head 1200, so that the nutrient storage part 1100 and the cartridge head 1200 are selectively screw-coupled to and separated from each other through screw coupling. It is obvious that the nutrient storage part 1100 and the cartridge head 1200 may be selectively coupled to and separated from each other in a different manner or may be provided as an integrated structure in which parts thereof cannot be coupled to or separated from each other.

The nutrient storage part 1100 may have a shape in which an internal space is formed and at least one surface thereof is open. As an example, the nutrient storage part 1100 may have a cylindrical shape in which an internal space is formed and the top surface thereof is open. The nutrients 1101 may be introduced into or discharged from the internal space of the nutrient storage part 1100 through the open surface of the nutrient storage part 1100.

The cartridge head 1200 may include a discharge tube 1210. The nutrients 1101 contained in the cartridge module 1000 may be discharged out of the cartridge module 1000 through the discharge tube 1210. When the cartridge module 1000 is mounted in the dispensing device, the nutrients 1101 contained in the cartridge module 1000 may be discharged through the discharge tube 1210 by the nutrient discharge operation of the dispensing device.

The cartridge module 1000 may include a cartridge identification unit that stores information about the cartridge module 1000.

The information about the cartridge module 1000 may include at least one of the serial code of the cartridge module 1000, the type or name of the nutrients 1101 contained in the cartridge module 1000, and the initially charged quantity information of the nutrients 1101.

The cartridge identification unit may be provided in the form of a memory chip 1300 as an example. The memory chip 1300 may be a non-volatile memory device. However, the cartridge identification unit is not necessarily limited to the memory chip, but may be provided in the form of a passive element such as a resistor, or in the form of one of an identification mark, an RFID tag, and an NFC tag. Hereinafter, for convenience of description, a case in which the cartridge identification unit is provided in the form of the memory chip 1300 will be described as an example.

When the cartridge identification unit is provided in the form of the memory chip 1300, the information about the cartridge module 1000 stored in the memory chip 1300 may include at least one of the serial code of the cartridge module 1000, the type or name of the nutrients 1101 contained in the cartridge module 1000, information about the initially charged quantity of nutrients 1101, information about the current residual quantity of nutrients 1101, a manufacturing date, and a manufacturer.

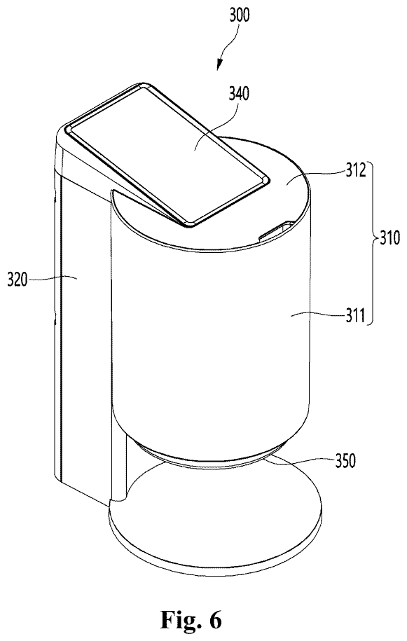

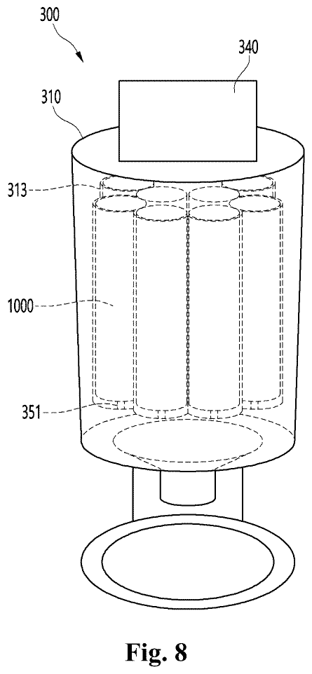

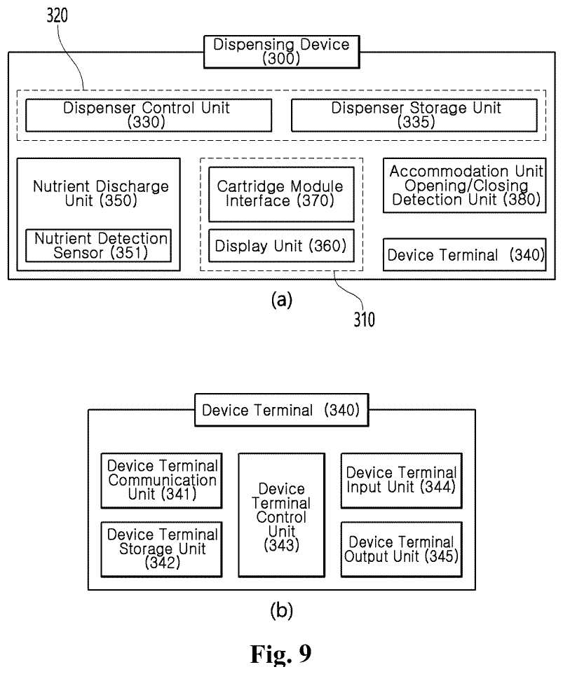

FIGS. 6 to 8 are diagrams of an example of the dispensing device 300 according to an embodiment, and FIG. 9 is a block diagram of the example of the dispensing device 300 according to the embodiment. The example of the dispensing device 300 will be described with reference to FIGS. 6 to 9 below.

The dispensing device 300 may include an accommodation unit 310, a main body 320, a dispenser control unit 330, a dispenser storage unit 335, a device terminal 340, and a nutrient discharge unit 350.

The accommodation unit 310 may accommodate one or more cartridge modules 1000.

The accommodation unit 310 may include a housing 311, a cover 312, and one or more cartridge insertion portions 313.

The cartridge insertion portions 313 may be located in the inner space of the housing 311.

The accommodation unit 310 may include a plurality of cartridge insertion portions 313. As an example, one cartridge module 1000 may be mounted in one of the cartridge insertion portions 313.

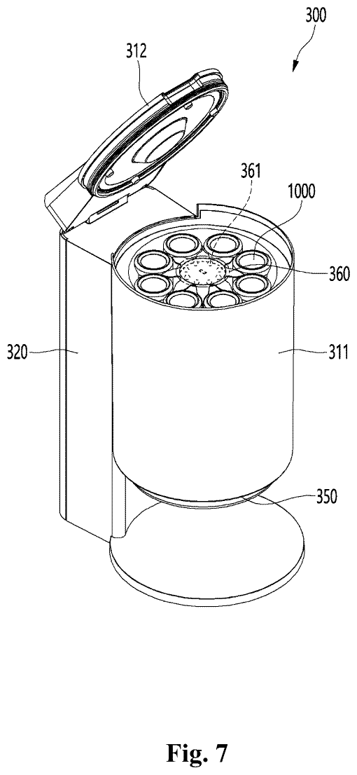

The accommodation unit 310 may be opened or closed by the relative movement of the cover 312 with respect to the housing 311. Referring to FIG. 6, when the housing 311 and the cover 312 are in a first positional relationship, the accommodation unit 310 may be closed. Referring to FIG. 7, when the housing 311 and the cover 312 are in a second positional relationship, the accommodation unit 310 may be opened. As an example, a user may open the accommodation unit 310 by moving the cover 312, may mount the cartridge modules 1000 in the cartridge insertion portions 313, and may close the accommodation unit 310 by moving the cover 312. As another example, a user opens the accommodation unit 310 by moving the cover 312, may remove the cartridge modules 1000 mounted in the cartridge insertion portions 313, may insert new cartridge modules 1000 into the cartridge insertion portions 313, and may close the accommodation unit 310 by moving the cover 312.

Although the cover 312 is shown as being located on the top of the housing 311 in FIGS. 6 to 8, this is only an example. The cover 312 may be located elsewhere, such as on the side or bottom of the housing 311.

Other components of the dispensing device 300 may be mounted in the main body 320. As an example, the dispenser control unit 330, which will be described later, may be mounted in the internal space of the main body 320.

The main body 320 may protect other components of the dispensing device 300. As an example, the main body 320 may protect the dispenser control unit 330, mounted therein, from external shock, etc.

Like the server control unit 130 described above, the dispensing device 300 may perform the processing and calculation of various types of information through the dispenser control unit 330. The dispenser control unit 330 may manage cartridge information about the cartridge modules 1000.

More specifically, as described above, each of the cartridge modules 1000 may be provided with a memory chip. When the cartridge modules 1000 are mounted in the dispensing device 300, the dispenser control unit 330 may obtain information about the cartridge modules 1000 from the memory chips provided for the respective cartridge modules 1000, and may transmit it to the server.

Furthermore, the dispenser control unit 330 may monitor the discharged quantity information of the nutrients discharged from the cartridge modules 1000 or the residual quantity information of the nutrients contained in the cartridge modules 1000. The discharged quantity information or residual quantity information may be output through the device terminal 340 or transmitted to the server or user terminal.

The dispenser control unit 330 may be electrically connected to the device terminal 340 and the nutrient discharge unit 350. In some cases, a display unit 360, a cartridge module interface 370, or an accommodation unit opening/closing detection unit 380, which will be described later, may be connected to the dispenser control unit 330.

Redundant descriptions of the parts of the dispenser control unit 330 that are the same as or similar to those of the server control unit 130 will be omitted.

Like the server storage unit 120 described above, the dispensing device 300 may store various types of data, programs, or applications necessary for operation in the dispenser storage unit 335. The dispenser storage unit 335 may store the information obtained by the dispensing device 300. The dispenser storage unit 335 may store cartridge information about the cartridge modules 1000. Redundant descriptions of the parts of the dispenser storage unit 335 that are the same as or similar to those of the server storage unit 120 will be omitted.

As shown in FIG. 9(b), the device terminal 340 may include a device terminal communication unit 341, a device terminal storage unit 342, a device terminal control unit 343, a device terminal input unit 344, and a device terminal output unit 345.

Like the terminal communication unit 410 of the user terminal 400 described above, the device terminal 340 may communicate with the outside through the device terminal communication unit 341. As an example, the device terminal communication unit 341 may communicate with the server 100 or user terminal 400. Redundant descriptions of the parts of the device terminal communication unit 341 that are the same as or similar to those of the terminal communication unit 410 will be omitted.

Like the terminal input unit 440 of the user terminal 400 described above, the device terminal 340 may obtain a user input through the device terminal input unit 344. Redundant descriptions of the parts of the device terminal input unit 344 that are the same as or similar to those of the terminal input unit 440 will be omitted.

Like the terminal output unit 450 of the user terminal 400 described above, the device terminal 340 may output information through the device terminal output unit 345. As an example, the device terminal 340 may output information related to the dispensing device 300 (e.g., residual quantity information, insufficient quantity information, etc.), the icon information intended to specify a user, the current state information of the user, or discharged quantity information through the device terminal output unit 345. Redundant descriptions of the parts of the device terminal output unit 345 that are the same as or similar to those of the terminal output unit 450 will be omitted.

Like the terminal storage unit 420 of the user terminal 400 described above, the device terminal 340 may store various types of data, programs, or applications necessary for operation in the device terminal storage unit 342. The device terminal storage unit 342 may store information obtained by the device terminal 340. Redundant descriptions of the parts of the device terminal storage unit 342 that are the same as or similar to those of the terminal storage unit 420 will be omitted.

Like the terminal control unit 430 of the user terminal 400 described above, the device terminal 340 may perform the processing and calculation of various types of information through the device terminal control unit 343. Redundant descriptions of the parts of the device terminal control unit 343 that are the same as or similar to those of the terminal control unit 430 will be omitted.

The dispensing device 300 may discharge the nutrients through the nutrient discharge unit 350.

The nutrient discharge unit 350 may include one or more nutrient detection sensors 351. The nutrient discharge unit 350 may include a plurality of nutrient detection sensors 351.

One nutrient detection sensor 351 may be provided to correspond to each of the cartridge insertion portions 313. As an example, a nutrient detection sensor 351 may be disposed in each of the cartridge insertion portions 313. One nutrient detection sensor 351 may be positioned to correspond to each of the cartridge modules 1000.

The dispensing device 300 may obtain discharges quantity information about the quantity of nutrients discharged from the cartridge modules 1000 through the nutrient detection sensors 351. As an example, the dispensing device 300 may count the number of nutrients discharged from the cartridge modules 1000 through the nutrient detection sensors 351.

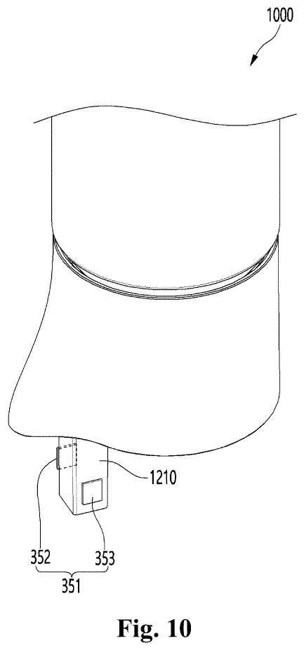

FIG. 10 is a diagram of an example of a nutrient detection sensor 351 according to an embodiment, which is a diagram of the example of the nutrient detection sensor 351 including a light emission unit 352 and a light reception unit 353.

The nutrient detection sensor 351 may include the light emission unit 352 and the light reception unit 353. Referring to FIG. 10, when the cartridge module 1000 is mounted in the dispensing device, the light emission unit 352 and the light reception unit 353 may be located on both sides of the discharge tube 1210 of the cartridge module 1000. The dispensing device 300 may generate discharged quantity information depending on whether the light output from the light emission unit 352 is received by the light reception unit 353. For example, when the light reception unit 353 does not receive the light output from the light emission unit 352, the dispensing device 300 may count one nutrient that has been dispensed.

In some embodiments, the nutrient discharge unit 350 includes discharge devices coupled to the cartridge modules 1000 and configured to discharge nutrients, drive motors mechanically coupled to the respective discharge devices and configured to operate the discharge devices, and nutrient detection sensors 351 provided under the respective discharge devices. Each of the plurality of drive motors may operate a discharge device coupled to a corresponding one of the plurality of cartridge modules 1000 in response to a control signal input from the dispenser control unit 330 to discharge one or more nutrients from the cartridge module 1000. The nutrient detection sensor 351 counts the number of nutrients discharged from the cartridge module 1000 to generate discharged quantity information, and the generated discharged quantity information may be provided to the dispenser control unit 330. When the nutrient discharge unit 350 is operated, the nutrient detection sensor 351 may detect and count the number of nutrients discharged from each cartridge module 1000, and the discharged quantity information generated by the counting may be transmitted to the device terminal 340 through the dispenser control unit 330. Thereafter, the device terminal 340 may transmit the discharged quantity information to the server 100 or user terminal 400.

The dispensing device 300 may further include a display unit 360.

The display unit 360 may be located in the internal space of the accommodation unit 310.

The display unit 360 may include one or more indication lamps 361. The display unit 360 may include a plurality of indication lamps 361. The indication lamps 361 may be LEDs as an example, but are not limited thereto.

One indication lamp 361 may be located in a portion corresponding to each of the cartridge insertion portions 313. One indication lamp 361 may be located in a portion corresponding to each of the cartridge modules 1000.

The display unit 360 may display information about the cartridge modules 1000. The dispensing device 300 may control the display unit 360 to display information about the cartridge modules 1000. More specific details regarding how the dispensing device 300 controls the display unit 360 will be described later.

The dispensing device 300 may further include one or more cartridge module interfaces 370. The dispensing device 300 may further include a plurality of cartridge module interfaces 370. Each of the cartridge module interface 370 may be provided in the form of an electrode, a terminal, or a combination thereof, but is not limited thereto.

One cartridge module interface 370 may correspond to each of the cartridge insertion units 313. As an example, the cartridge module interfaces 370 may be disposed in the respective cartridge insertion portions 313. One cartridge module interface 370 may correspond to each of the cartridge modules 1000. Each of the cartridge module interfaces 370 may be located at a portion in contact with the cartridge identification unit (e.g., the memory chip) of the cartridge module 1000 within the cartridge insertion portion 313.

The dispensing device 300 may be electrically connected to the cartridge module 1000 through the cartridge module interface 370. As an example, the dispensing device 300 may be electrically connected to the cartridge identification unit (e.g., the memory chip) of the cartridge module 1000 through the cartridge module interface 370.

The cartridge module interface 370 may obtain information about the cartridge module by reading the information of the cartridge identification unit. When the cartridge identification unit is provided in the form of a memory chip, updated information about the cartridge module may be written to the memory chip.

As an example, the dispensing device 300 may obtain or read the information about the cartridge module 1000 stored in the memory chip of the cartridge module 1000 through the cartridge module interface 370.

The dispensing device 300 may write information to the cartridge module 1000 through the cartridge module interface 370. As an example, the dispensing device 300 may write information to the memory chip of the cartridge module 1000 through the cartridge module interface 370.

The dispensing device 300 may detect whether the cartridge module 1000 has been mounted in the cartridge insertion portion 313 through the cartridge module interface 370. For example, when the dispensing device 300 is electrically connected to the cartridge module 1000, it may be determined that the cartridge module 1000 has been mounted. As a more specific example, when the dispensing device 300 is electrically connected to the memory chip of the cartridge module 1000, it may be determined that the cartridge module 1000 has been mounted. It is obvious that the dispensing device 300 may include a sensor configured to detect whether the cartridge module 1000 has been mounted in the cartridge insertion portion 313 separately from the cartridge module interface 370.

The dispensing device 300 may further include the accommodation unit opening/closing detection unit 380. The dispensing device 300 may detect whether the accommodation unit is opened or closed through the accommodation unit opening/closing detection unit 380. As an example, the dispenser control unit 330 obtains an accommodation unit opening/closing signal, indicating whether the accommodation unit is opened or closed, from the accommodation unit opening/closing detection unit 380, and detects whether the accommodation unit is opened or closed based on the obtained accommodation unit opening/closing signal.

A method of managing cartridge modules will be described in more detail below.

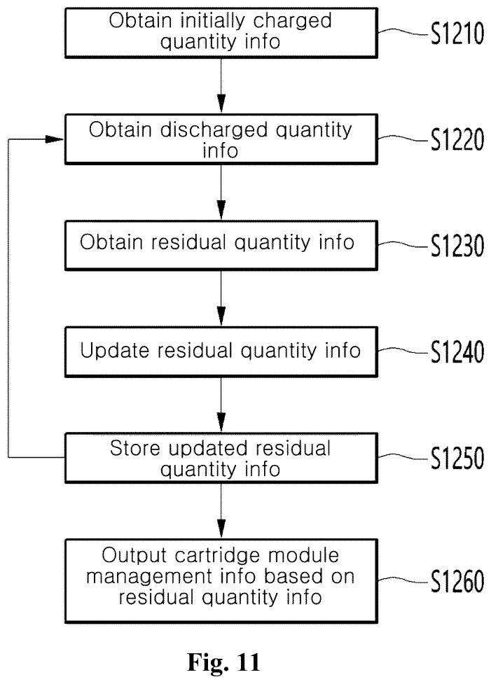

FIG. 11 is a flowchart of an example of a method of managing cartridge modules according to an embodiment, which is a flowchart of a residual quantity management method, which is a method of managing the residual quantity of nutrients contained in cartridge modules. Not all of the steps of FIG. 11 are essential, and the sequence of the steps may be changed or at least some of the steps may be omitted. For example, step S1230 of obtaining residual quantity information may be performed before step S1220 of obtaining discharged quantity information. As another example, step S1220 of obtaining discharged quantity information and step S1230 of obtaining residual quantity information may be performed simultaneously.

The residual quantity management method may include step S1210 of obtaining initially charged quantity information.

The initially charged quantity information may be obtained for each of the cartridge modules. For example, when N cartridge modules are mounted in the dispensing device, N pieces of initially charged quantity information may be obtained.

According to an embodiment, the initially charged quantity information may be stored in the memory chip of the cartridge module. When a cartridge module is mounted in the dispensing device, the dispensing device may obtain the initially charged quantity information from the memory chip of the mounted cartridge module. In some cases, the dispensing device may transmit the obtained initially charged quantity information to at least one of the server and the user terminal.

According to another embodiment, the cartridge module identification information that can identify the cartridge module is displayed on the cartridge module, and a user obtains cartridge module identification information through the user terminal, the dispensing device, or other devices, so that at least one of the server, the user terminal, and the dispensing device may obtain initially charged quantity information.

As an example, the cartridge module identification information may be a code that can identify the cartridge module, such as a barcode or QR code. In this case, at least one of the server, the user terminal, and the dispensing device may obtain initially charged quantity information in such a manner that a user scans a code through the user terminal, the dispensing device, or other devices (e.g., a barcode reader, a QR code reader, etc.).

As another example, the cartridge module identification information may be a serial number that can identify the cartridge module. In this case, at least one of the server, the user terminal, and the dispensing device may obtain initially charged quantity information in such a manner that a user enters a serial number into the user terminal, the dispensing device, or other devices (e.g., a tablet PC, a computer, a notebook computer, etc.).

In this case, the cartridge module identification information does not necessarily need to be displayed on the cartridge module. The cartridge module identification information may be provided in another manner in which the cartridge module identification information can be recognized as cartridge module identification information corresponding to a specific cartridge module, as in the case where the cartridge module identification information is displayed on a separate printed material provided together with the cartridge module, the packaging of the cartridge module, or the like.

The residual quantity management method may include step S1220 of obtaining discharged quantity information.

As described above, when one or more nutrients are discharged from the cartridge module mounted in the dispensing device, the dispensing device may obtain information about the quantity of discharged nutrients. In some cases, the dispensing device may transmit the obtained discharged quantity information to at least one of the server and the user terminal.

The discharged quantity information may be obtained for each of the cartridge modules. For example, when N cartridge modules are mounted in the dispensing device, N pieces of discharged quantity information may be obtained.

The residual quantity management method may include step S1230 of obtaining residual quantity information.

In the case of a cartridge module newly installed in the dispensing device, the residual quantity information may be the same as the initially charged quantity information.

According to an embodiment, the residual quantity information may be stored in the memory chip of the cartridge module. The dispensing device may obtain the residual quantity information from the memory chip of the mounted cartridge module. In some cases, the dispensing device may transmit the obtained residual quantity information to at least one of the server and the user terminal.

According to another embodiment, the residual quantity information may be stored in the dispensing device. In this case, the dispensing device obtaining residual quantity information may mean loading the stored residual quantity information. In some cases, the dispensing device may transmit the stored residual quantity information to at least one of the server and the user terminal.

According to still another embodiment, the residual quantity information may be stored on the server. In this case, the server obtaining residual quantity information may mean loading the stored residual quantity information. In some cases, the server may transmit the stored residual quantity information to at least one of the dispensing device and the user terminal.

The residual quantity management method may include step S1240 of updating the residual quantity information.

The residual quantity information may be updated by subtracting the discharged quantity information obtained in step S1220 from the residual quantity information obtained in step S1230. At least one of the dispensing device, the server, and the user terminal may perform step S1240 of updating the residual quantity information and transmit the updated residual quantity information to another component. As an example, the dispensing device may perform step S1240 of updating the residual quantity information and transmit the updated residual quantity information to at least one of the server and the user terminal.

As shown in FIG. 11, steps S1220, S1230, S1240, and S1250 may be performed repeatedly.

The residual quantity management method may include step S1250 of storing the updated residual quantity information.

In the case of an embodiment in which residual quantity information is stored in the memory chip of the cartridge module, updated residual quantity information may be stored in the memory chip of the cartridge module.

In the case of an embodiment in which residual quantity information is stored in the dispensing device, updated residual quantity information may be stored in the dispensing device.

In the case of an embodiment in which residual quantity information is stored in the server, updated residual quantity information may be stored in the server.

The residual quantity management method may include step S1260 of outputting cartridge module management information based on the residual quantity information.

At least one of the dispensing device, the server, and the user terminal may generate cartridge module management information based on the residual quantity information. The dispensing device or user terminal may output the generated cartridge module management information. As an example, the server may generate cartridge module management information based on the residual quantity information and transmit the generated cartridge module management information to the dispensing device or user terminal, and the dispensing device or user terminal may output the received cartridge module management information.

The cartridge module management information may include the residual quantity information.

The cartridge module management information may include insufficient quantity information. The insufficient quantity information may be information indicating that the residual quantity of nutrients contained in the cartridge module is smaller than a reference value.

The cartridge module management information may include expected runout time information. The expected runout time information may be information indicating the period in which the nutrients contained in the cartridge module is expected to run out. A user may schedule the time to order a new cartridge module or the time to replace the cartridge module by checking the expected runout time information.

The expected runout time information may be calculated by reflecting the runout speed of the nutrients therein. As an example, the runout speed may be calculated based on changes in information about the quantity of residual nutrients, and the expected runout time information may be calculated using the calculated runout speed and the current residual quantity information.

The cartridge module management information may include cartridge module mounting information. The cartridge module mounting information may be information indicating whether a cartridge module has been mounted in the dispensing device.

The cartridge module management information may include cartridge module sales information. Although the cartridge module sales information may include the types or prices of one or more cartridge modules available for purchase or a combination thereof, it is not limited thereto.

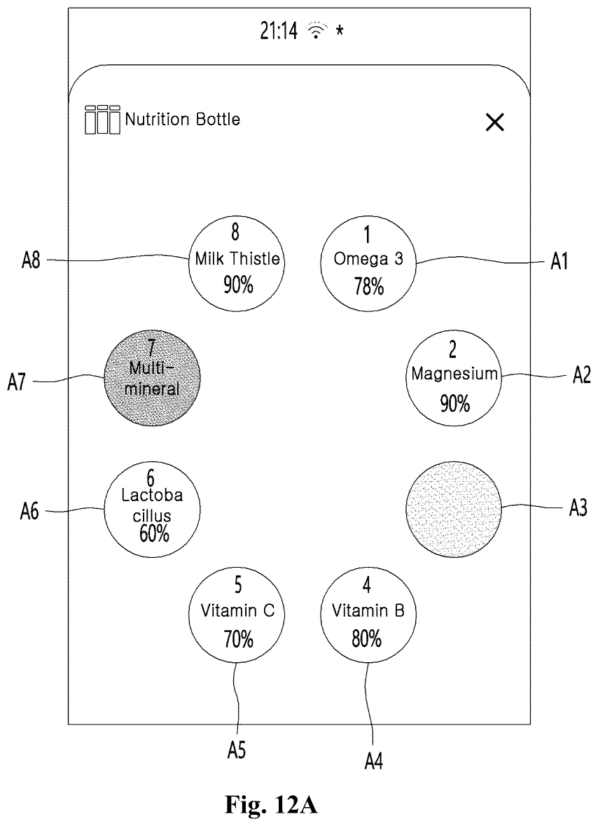

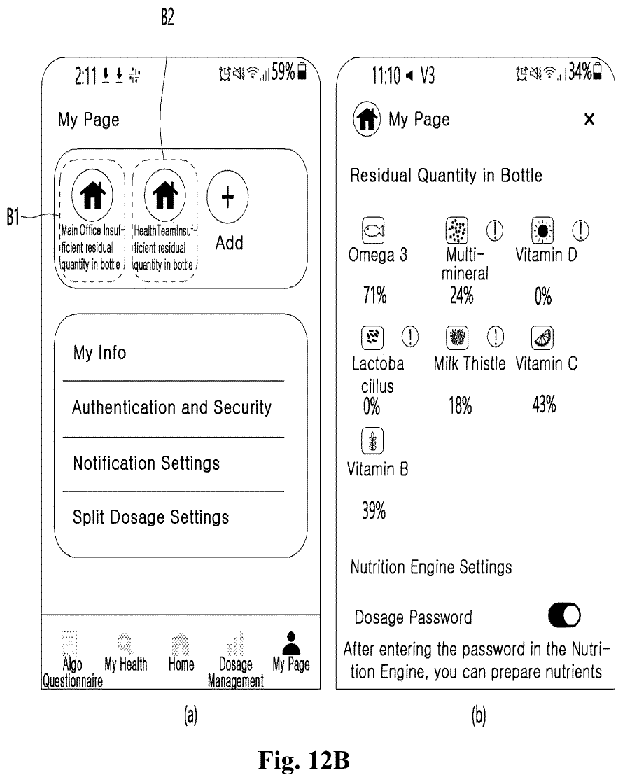

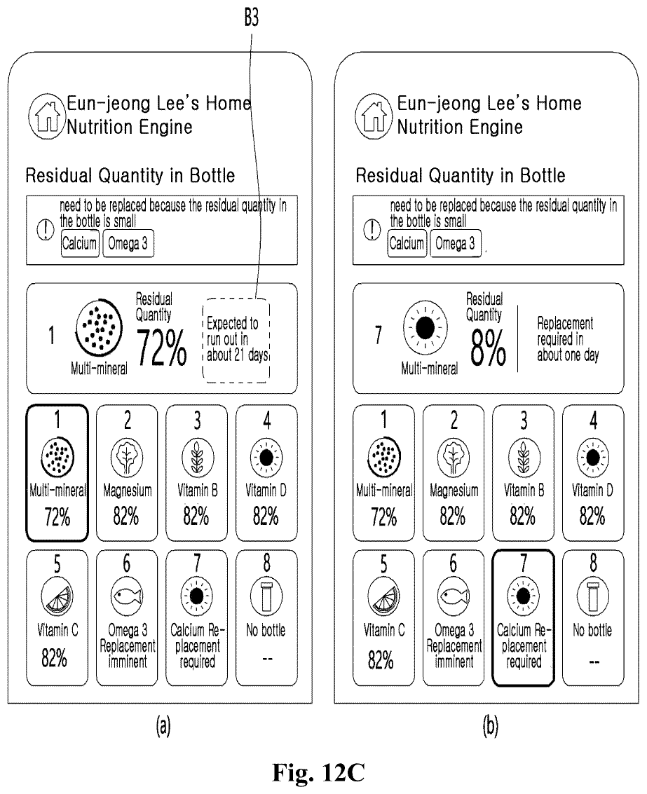

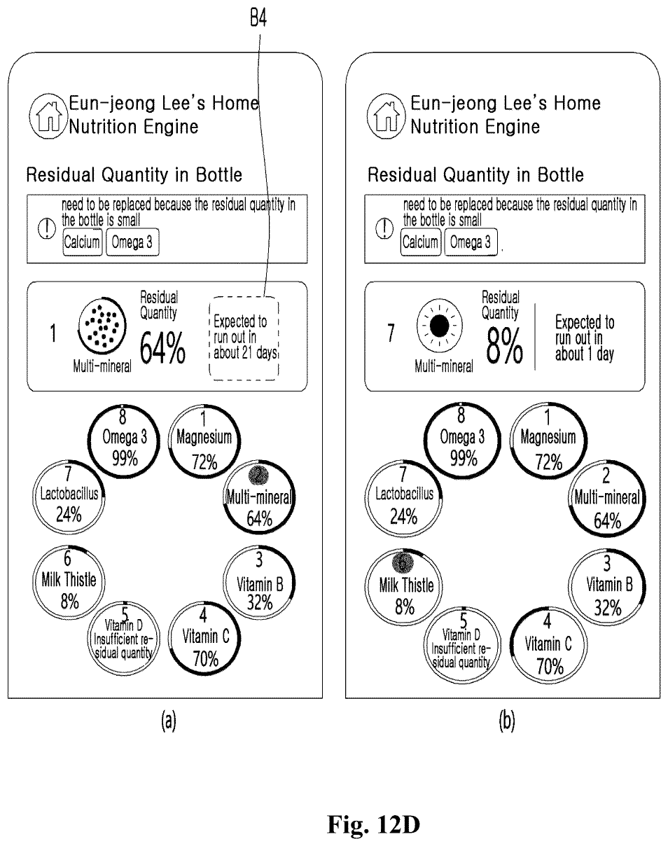

FIG. 12a is a diagram of a first example of displaying cartridge module management information, which is an example of outputting cartridge module management information according to an embodiment.

The user terminal or dispensing device may display a screen containing information about the cartridge insertion portions or cartridge modules. The screen may include a plurality of area images arranged to correspond to the cartridge insertion portions or cartridge modules. The user terminal or dispensing device may display information about the cartridge insertion portions or cartridge modules, corresponding to the areas, in the areas.

The user terminal or dispensing device may display a screen containing residual quantity information for each of the cartridge modules mounted in the dispensing device. As an example, referring to FIG. 12a, the user terminal or dispensing device displays the residual quantities of cartridge modules, corresponding to the first, second, and fourth to eighth areas A1, A2, and A4 to A8, on the first, second, and fourth to eighth areas A1, A2, and A4 to A8, respectively. In this case, the first, second, and fourth to eighth areas A1, A2, and A4 to A8 on the display may be arranged to correspond to the arrangement of the first to N-th cartridge modules in the dispensing device shown in FIG. 7. The number of slot into which each of the first to N-th cartridge modules is inserted and information about the cartridge module (e.g., the type or name of nutrients stored, current residual quantity information, an expiration date, etc.) may be displayed in a corresponding one of the individual areas A1 to A8.

The user terminal or dispensing device may display insufficient quantity information for a cartridge module whose residual quantity is smaller than the reference value. As an example, referring to FIG. 12a, when the residual quantity of the cartridge module mounted in the seventh cartridge insertion portion is smaller than the reference value, the user terminal or dispensing device may display the seventh area A7 on the display in a color different from that of the remaining areas A1 to A6 and A8, or may display information about the residual quantity in an emphasized manner.

For example, when the reference value is set to 30% and the residual quantity of nutrients in the cartridge module is 30% or smaller, the area A7 representing cartridge module No. 7 may be displayed in a color (e.g., red) different from the color (e.g., gray) of other areas as shown in FIG. 12a, or the color, thickness, or size of a text indicating the residual quantity may be displayed differently from that of other cartridge modules. Alternatively, when the residual quantity is equal to or smaller than the reference value, information about an insufficient quantity may be displayed together with exclamation marks as shown in FIG. 12b, together with a text such as ‘replacement required’ or ‘replacement imminent’ as shown in FIG. 12c, or a text such as ‘expected to run out in 1 day’ as shown in FIG. 12d.

The user terminal or dispensing device may display the cartridge insertion portion mounted with the cartridge module and the cartridge insertion portion not mounted with the cartridge module in different manners based on the cartridge module mounting information. As an example, referring to FIG. 12a, the user terminal or dispensing device displays the third area A3, corresponding to the third cartridge insertion portion in which the cartridge module is not mounted, in a color different from the color of the remaining areas A1, A2, A4 to A8.

Although cartridge module management information is illustrated as being output from the user terminal or dispensing device in FIG. 12a, the cartridge module management information may also be output through other devices.

FIG. 12b is a diagram of a second example of displaying cartridge module management information according to an embodiment.

The user terminal may display a screen including an area corresponding to one or more dispensing devices registered for use by a user. As an example, referring to FIG. 12b, the user terminal may display information related to a first dispensing device in a first area B1 and display information related to a second dispensing device in a second area B2 on the display thereof.

When obtaining an input intended to select an area corresponding to a specific dispensing device, the user terminal may display the cartridge module management information of the specific dispensing device. As an example, referring to FIG. 12b, when obtaining an input intended to select an area corresponding to the first dispensing device, the user terminal may display the cartridge module management information of the first dispensing device. In drawing (b) of FIG. 12b, pieces of residual quantity information are displayed for respective cartridge modules, and pieces of insufficient quantity information are also displayed for some cartridge modules such as cartridge modules for multi-mineral, vitamin D, lactic acid bacteria, and milk thistle.

Although the user terminal is illustrated as displaying the cartridge module management information in FIG. 12b, the cartridge module management information may be displayed through a device other than the user terminal, such as the dispensing device.

FIG. 12c is a diagram of a third example of displaying cartridge module management information according to an embodiment. In this case, the following description will be given with a focus on expected runout time information.

The user terminal or dispensing device may display a screen including expected runout time information. As an example, referring to drawing (a) of FIG. 12c, the user terminal or dispensing device may display the expected runout time information of a specific cartridge module in a first area B3 on the display thereof.

The user terminal or dispensing device may display expected runout time information in different manners depending on the expected runout time information. As an example, the user terminal or dispensing device may display the expected runout time information of a cartridge module requiring replacement differently from the expected runout time information of a cartridge module not requiring replacement. As a more specific example, referring to FIG. 12c, the user terminal or dispensing device may display the expected runout time information of the cartridge module requiring replacement in a color different from the color of the expected runout time information of the cartridge module not requiring replacement.

FIG. 12d is a diagram of a fourth example of displaying cartridge module management information according to an embodiment. In this case, the following description will be given with a focus on expected runout time information.

The user terminal or dispensing device may display a screen including expected runout time information. As an example, referring to drawing (a) of FIG. 12d, the user terminal or dispensing device may display the expected runout time information of a specific cartridge module in a first area B4 on the display thereof.

The user terminal or dispensing device may display expected runout time information in different manners depending on the expected runout time information. As an example, the user terminal or dispensing device may display the expected runout time information of a cartridge module requiring replacement differently from the expected runout time information of a cartridge module not requiring replacement. As a more specific example, referring to FIG. 12d, the user terminal or dispensing device may display the expected runout time information of a cartridge module requiring replacement in a color different from the color of the expected runout time information of a cartridge module not requiring replacement.

Although the cartridge module management information is illustrated as being output from the user terminal or dispensing device in FIGS. 12c and 12d, the cartridge module management information may be output through other devices.

The cartridge module management information display method including the residual quantity information, the insufficient quantity information, the cartridge module mounting information, and the expected runout time information shown in FIG. 12 is only an example, and the cartridge module management information may be displayed in a manner different from that of FIG. 12.

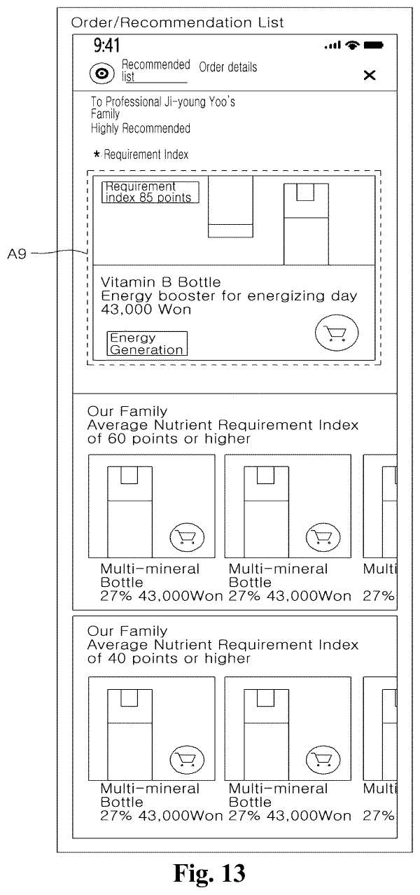

FIG. 13 is a diagram of another example of displaying cartridge module management information, which is an example of outputting cartridge module management information according to an embodiment.

The user terminal or dispensing device may display a screen containing cartridge module sales information. The screen may include an area corresponding to a cartridge module. The user terminal or dispensing device may display the cartridge module sales information of the cartridge module, corresponding to the area, in the area.

The user terminal or dispensing device may display a screen containing information about the type of cartridge module available for purchase. As an example, referring to FIG. 13, the user terminal or dispensing device displays information about a vitamin B cartridge module in an area A9 on the display thereof.

The user terminal or dispensing device may display a screen containing information about the price of a cartridge module available for purchase. As an example, referring to FIG. 13, the user terminal or dispensing device displays information about the price of a vitamin B cartridge module in the area A9 on the display thereof.

The method of displaying cartridge module sales information shown in FIG. 13 is merely an example, and the cartridge module sales information may be displayed by a method different from that of FIG. 13.

Although the cartridge module management information is illustrated as being output from the user terminal or dispensing device in FIG. 13, the cartridge module management information may be output through a device other than the user terminal or dispensing device.

Although not shown in the drawings, the residual quantity management method may further include the step of obtaining cartridge module purchase information. Although the cartridge module purchase information may include the type of cartridge module that a user wants to purchase, it is not limited thereto. As an example, the user terminal or dispensing device may receive cartridge module purchase information from a user and transmit the input cartridge module purchase information to the server. Accordingly, the user can purchase a new cartridge module.

The above-described residual quantity management method may be performed by the system. Some examples in which the server, the user terminal, and the dispensing device perform a residual quantity management method while operating in conjunction with each other will be described below. The server, the user terminal, and the dispensing device may operate in conjunction with each other by a method different from those of the examples to be described below.

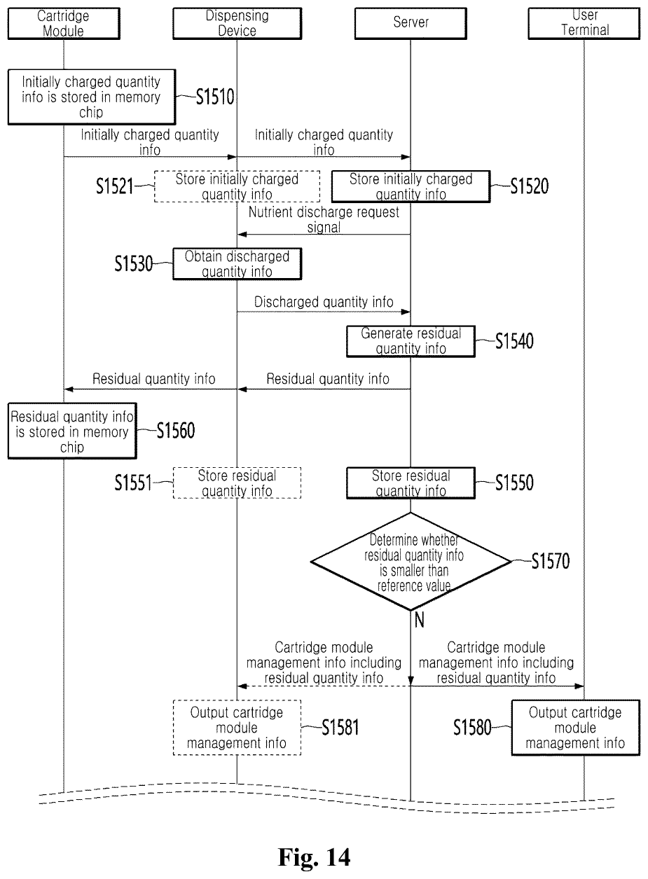

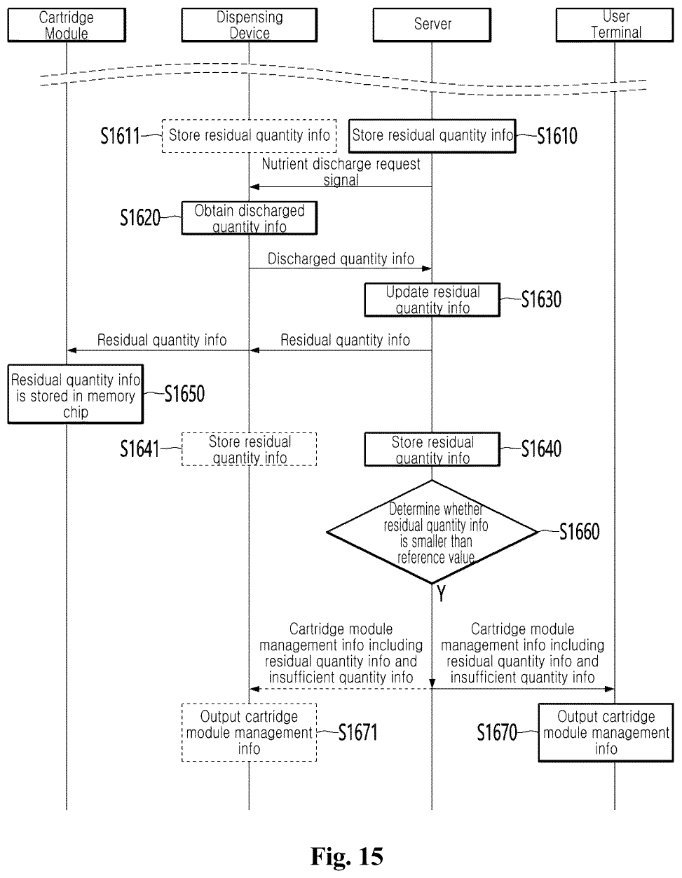

FIGS. 14 and 15 are flowcharts of a first embodiment in which the server, the user terminal, and the dispensing device perform a residual quantity management method while operating in conjunction with each other, wherein FIG. 14 is a flowchart of a case in which a cartridge module is newly installed in the dispensing device, and FIG. 15 is a flowchart of a case that is performed after the cartridge module has been used. Referring to FIGS. 14 and 15, the cartridge module may include a memory chip configured to store initially charged quantity information and residual quantity information, the server may generate or update residual quantity information based on discharged quantity information, and the information included in the cartridge module management information may vary depending on whether the residual quantity information is smaller than a reference value.

Referring to FIG. 14, initially charged quantity information may be stored in the memory chip of the cartridge module as information about the cartridge module in step S1510. In addition to information about the initially charged quantity of nutrients accommodated in the cartridge module, the information about the cartridge module may include the identification information of the cartridge module (e.g., the serial number of the cartridge module), information about the nutrients accommodated in the cartridge module (e.g., the type or name of nutrients), information about the current residual quantity of nutrients accommodated in the cartridge module, the manufacturing date and time of the nutrients, the expiration date of the nutrients, etc. When the cartridge module is first inserted into the insertion portion, the initially charged quantity information and the residual quantity information may be stored as the same value.

When the cartridge module is inserted into the insertion portions 313, the dispensing device may obtain information about the cartridge module, including initially charged quantity information, from the cartridge module. When obtaining the information about the cartridge module, the dispensing device may detect one of the plurality of insertion portions 313 into which the cartridge module is inserted, may generate information about the detected slot, and may store and process the information about the cartridge module and the information about the slot in a bundle. The following description will be made on the assumption that the information about the cartridge module is processed together with the information about the slot.

The dispensing device may transmit the information about the cartridge module, including the obtained initially charged quantity information, to the server. The server may store the information about the cartridge module, including the obtained initially charged quantity information, in step S1520. As an example, the server may store initially charged quantity information for each cartridge module. In some cases, the dispensing device may store initially charged quantity information in step S1521. The server may transmit a nutrient discharge request signal for each cartridge module to the dispensing device.

The dispensing device may discharge one or more nutrients for each cartridge module in response to the nutrient discharge request signal.

As the nutrients are discharged, the dispensing device may obtain discharged quantity information through the nutrient detection sensor 351 provided for each cartridge module in step S1530.

The dispensing device may transmit the discharged quantity information, obtained for each cartridge module, to the server.

The server may generate residual quantity information for each cartridge module based on the discharged quantity information obtained for each cartridge module in step S1540. The server may store the residual quantity information for each cartridge module in step $1550.

The server may transmit the residual quantity information for each cartridge module to the dispensing device. The dispensing device may store the received residual quantity information in the memory chip of each cartridge module. Accordingly, the residual quantity information may be stored in the memory chip of each cartridge module in step S1560.

In some cases, the dispensing device may store the residual quantity information in step S1551.

The server may determine whether the residual quantity information for each cartridge module is smaller than the reference value in step S1570. When it is determined that the residual quantity information is not smaller than the reference value, the server may transmit the cartridge module management information, including the residual quantity information, and the information about the slot to the user terminal. In some cases, the server may transmit the cartridge module management information, including the residual quantity information, and the information about the slot to the dispensing device.

The user terminal may output the received cartridge module management information in step S1580. Accordingly, the user may check the residual quantity of nutrients for each cartridge module.

In some cases, the dispensing device may output the received cartridge module management information in step S1581. Accordingly, the user may check the residual quantity of nutrients for each cartridge module.

Referring to FIG. 15, the server may store information about the residual quantity of the used cartridge module in step S1610. In some cases, the dispensing device may store information about the residual quantity of the used cartridge module in step S1611.

The server may transmit a nutrient discharge request signal for each cartridge module to the dispensing device.

The dispensing device may discharge one or more nutrients in response to the nutrient discharge request signal for each cartridge module.

As the nutrients are discharged, the dispensing device may obtain discharged quantity information for each cartridge module in step S1620.

The dispensing device may transmit the obtained discharged quantity information to the server.

The server may update the residual quantity information for each cartridge module based on the obtained discharged quantity information in step S1630. The server may store the updated residual quantity information for each cartridge module in step S1640.

The server may transmit the updated residual quantity information to the dispensing device. The dispensing device may store the received residual quantity information in the memory chip of the cartridge module. Accordingly, the updated residual quantity information may be stored in the memory chip of the cartridge module in step S1650.

In some cases, the dispensing device may store the updated residual quantity information in step S1641.

The server may determine whether the residual quantity information for each cartridge module is smaller than the reference value in step S1660. When it is determined that the residual quantity information is smaller than the reference value, the server may transmit cartridge module management information including residual quantity information and insufficient quantity information for each cartridge module to the user terminal. In some cases, the server may transmit the cartridge module management information including residual quantity information and insufficient quantity information to the dispensing device.

The user terminal may output the received cartridge module management information in step S1670. Accordingly, the user may check the residual quantity of nutrients for each cartridge module and a cartridge module having an insufficient residual quantity.

In some cases, the dispensing device may output the received cartridge module management information in step S1671. Accordingly, the user may check the residual quantity of nutrients for each cartridge module and a cartridge module having an insufficient residual quantity.

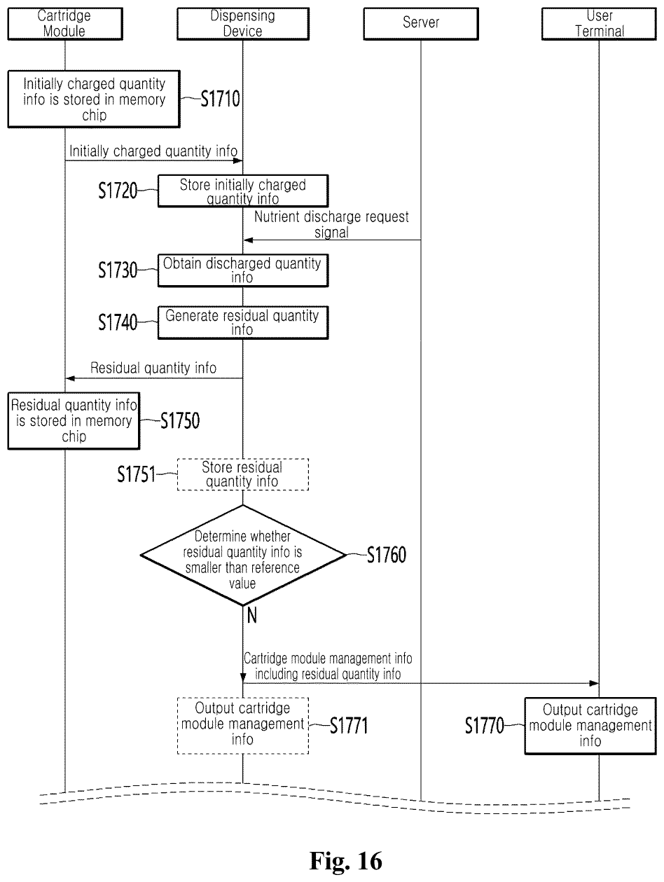

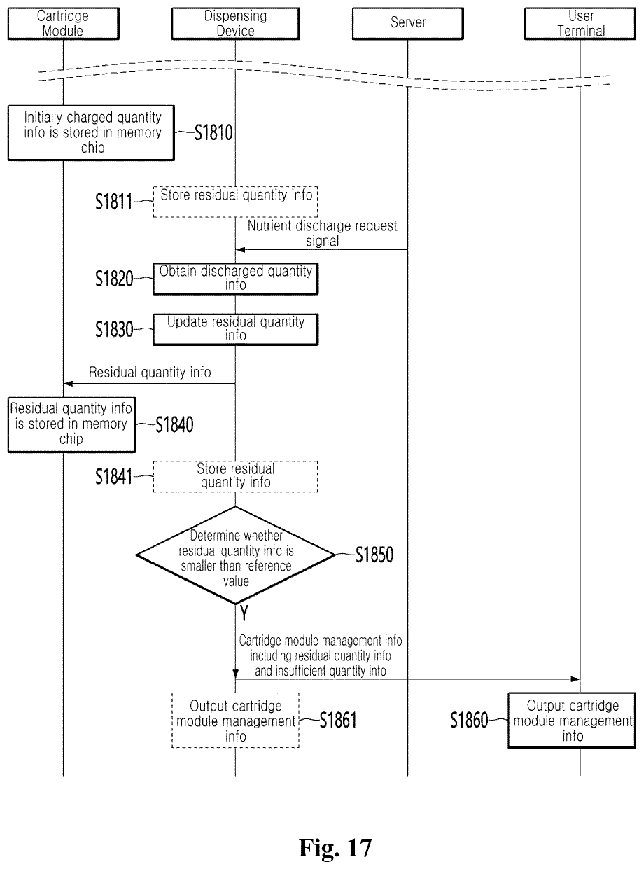

FIGS. 16 and 17 are flowcharts of a second embodiment in which the server, the user terminal, and the dispensing device perform a residual quantity management method while operating in conjunction with each other, wherein FIG. 16 is a flowchart of a case in which a cartridge module is newly installed in the dispensing device and FIG. 17 is a flowchart of a case that is performed after the cartridge module has been used. Referring to FIGS. 16 and 17, the cartridge module may include a memory chip configured to store initially charged quantity information and residual quantity information, the dispensing device may generate or update residual quantity information based on the discharged quantity information, and the information included in the cartridge module management information may vary depending on whether the residual quantity information is smaller than the reference value.

Referring to FIG. 16, the initially charged quantity information may be stored in the memory chip of the cartridge module in step S1710.

The dispensing device may obtain the initially charged quantity information from the cartridge module.

The dispensing device may store the initially charged quantity information in step S1720. As an example, the dispensing device may store initially charged quantity information for each cartridge module.

The dispensing device may receive a nutrient discharge request signal from the server.

The dispensing device may discharge one or more nutrients in response to the nutrient discharge request signal.

As the nutrients are discharged, the dispensing device may obtain discharged quantity information in step S1730.

The dispensing device may generate residual quantity information based on the obtained discharged quantity information in step S1740.

The dispensing device may store the residual quantity information in the memory chip of the cartridge module. Accordingly, the residual quantity information may be stored in the memory chip of the cartridge module in step S1750. In some cases, the dispensing device may store the residual quantity information in step S1751.

The dispensing device may determine whether the residual quantity information is smaller than the reference value in step S1760. When it is determined that the residual quantity information is not smaller than the reference value, the dispensing device may transmit cartridge module management information including the residual quantity information to the user terminal.

The user terminal may output the received cartridge module management information in step S1770. Accordingly, the user may check the residual quantity of nutrients for each cartridge module.

In some cases, the dispensing device may output the cartridge module management information in step S1771. Accordingly, the user may check the residual quantity of nutrients for each cartridge module.

Referring to FIG. 17, the residual quantity information may be stored in the memory chip of the cartridge module in step S1810. In some cases, the dispensing device may store information about the residual quantity of the used cartridge module in step S1811.

The dispensing device may receive a nutrient discharge request signal from the server.

The dispensing device may discharge one or more nutrients in response to the nutrient discharge request signal.

As the nutrients are discharged, the dispensing device may obtain discharged quantity information in step S1820.

The dispensing device may update residual quantity information based on the obtained discharged quantity information in step S1830.

The dispensing device may store the residual quantity information in the memory chip of the cartridge module. Accordingly, the updated residual quantity information may be stored in the memory chip of the cartridge module in step S1840. In some cases, the dispensing device may store the updated residual quantity information in step S1841.

The dispensing device may determine whether the residual quantity information is smaller than the reference value in step S1850. When it is determined that the residual quantity information is smaller than the reference value, the dispensing device may transmit cartridge module management information including the residual quantity information and insufficient quantity information to the user terminal.

The user terminal may output the received cartridge module management information in step S1860. Accordingly, the user may determine the residual quantity of nutrients for each cartridge module and a cartridge module having an insufficient residual quantity.

In some cases, the dispensing device may output the cartridge module management information in step S1861. Accordingly, a user may determine the residual quantity of nutrients for each cartridge module and the cartridge module having an insufficient residual quantity.

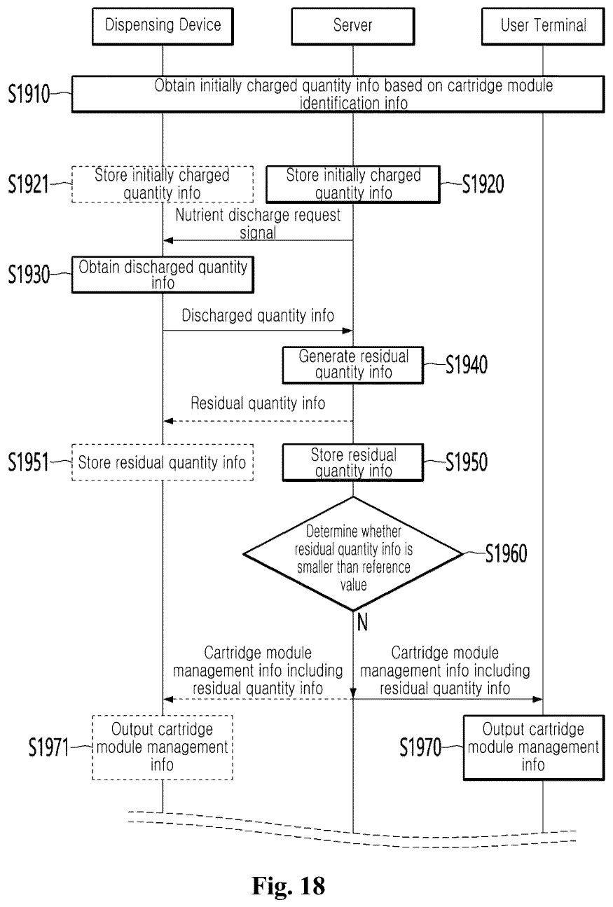

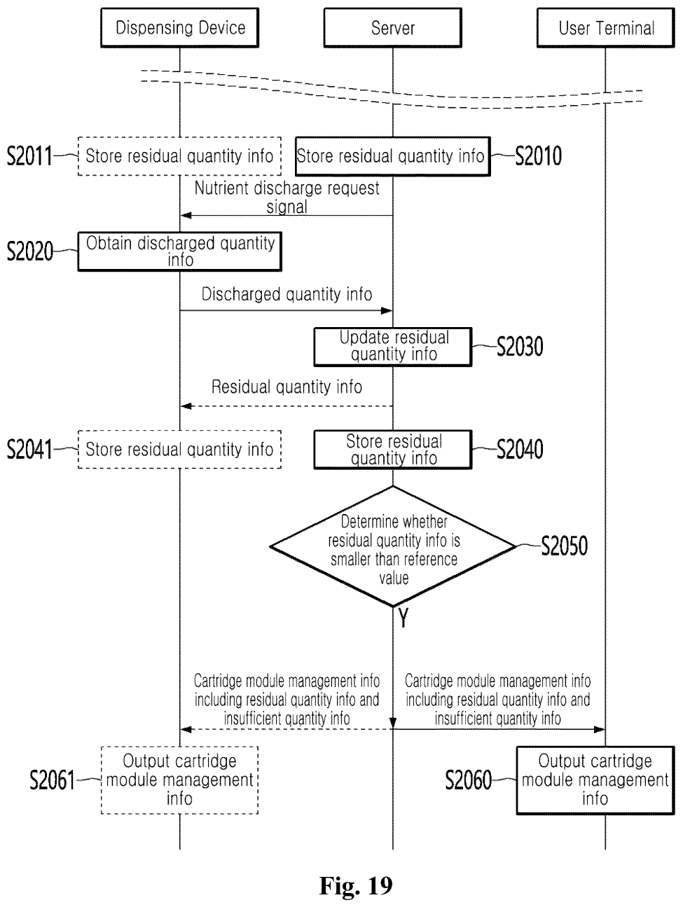

FIGS. 18 and 19 are flowcharts of a third embodiment in which the server, the user terminal, and the dispensing device perform a residual quantity management method while operating in conjunction with each other, wherein FIG. 18 is a flowchart of a case where a cartridge module is newly installed in the dispensing device and FIG. 19 is a flowchart of a case that is performed after a cartridge module has been used. Referring to FIGS. 18 and 19, at least one of the server, the user terminal, and the dispensing device may obtain initially charged quantity information based on cartridge identification information, the server may create or update residual quantity information based on the discharged quantity information, and the information included in the cartridge module management information may vary depending on whether the residual quantity information is smaller than the reference value.

Referring to FIG. 18, at least one of the server, the user terminal, and the dispensing device may obtain initially charged quantity information based on cartridge identification information in step S1910. As an example, the server may obtain cartridge module identification information and obtain initially charged quantity information based on the obtained cartridge module identification information. As another example, the user terminal or dispensing device may obtain cartridge module identification information and transmit the obtained cartridge module identification information to the server, and the server may obtain initially charged quantity information based on the received cartridge module identification information. As still another example, the user terminal or dispensing device may obtain cartridge module identification information, obtain initially charged quantity information based on the obtained cartridge module identification information, and transmit the obtained initially charged quantity information to the server.

The server may store the initially charged quantity information in step S1920. In some cases, the dispensing device may store the initially charged quantity information in step S1921.

The server may transmit a nutrient discharge request signal to the dispensing device. The dispensing device may discharge one or more nutrients in response to the nutrient discharge request signal.

As the nutrients are discharged, the dispensing device may obtain discharged quantity information in step S1930.

The dispensing device may transmit the obtained discharged quantity information to the server.

The server may generate residual quantity information based on the obtained discharged quantity information in step S1940. The server may store the residual quantity information in step S1950.

In some cases, the server may transmit the residual quantity information to the dispensing device. In this case, the dispensing device may store the residual quantity information in step S1951.

The server may determine whether the residual quantity information is smaller than the reference value in step S1960. When it is determined that the residual quantity information is not smaller than the reference value, the server may transmit cartridge module management information including the residual quantity information to the user terminal. In some cases, the server may transmit cartridge module management information including the residual quantity information to the dispensing device.

The user terminal may output the received cartridge module management information in step S1970. Accordingly, a user may determine the residual quantity of nutrients for each cartridge module.

In some cases, the dispensing device may output the received cartridge module management information in step S1971. Accordingly, a user may determine the residual quantity of nutrients for each cartridge module.

Referring to FIG. 19, the server may store the residual quantity information of the used cartridge module in step S2010. In some cases, the dispensing device may store the residual quantity information of the used cartridge module in step S2011.

The server may transmit a nutrient discharge request signal to the dispensing device.

The dispensing device may discharge one or more nutrients in response to the nutrient discharge request signal.

As the nutrients are discharged, the dispensing device may obtain discharged quantity information in step S2020.

The dispensing device may transmit the obtained discharged quantity information to the server.

The server may update the residual quantity information based on the obtained discharged quantity information in step S2030. The server may store the updated residual quantity information in step S2040.

In some cases, the server may transmit the updated residual quantity information to the dispensing device. In this case, the dispensing device may store the updated residual quantity information in step S2041.

The server may determine whether the residual quantity information is smaller than the standard value in step S2050. When it is determined that the residual quantity information is smaller than the reference value, the server may transmit cartridge module management information, including the residual quantity information and insufficient quantity information, to the user terminal. In some cases, the server may transmit cartridge module management information, including the residual quantity information and insufficient quantity information, to the dispensing device.

The user terminal may output the received cartridge module management information in step S2060. Accordingly, the user may determine the residual quantity of nutrients for each cartridge module and a cartridge module having an insufficient residual quantity.

In some cases, the dispensing device may output the received cartridge module management information in step S2061. Accordingly, the user may determine the residual quantity of nutrients for each cartridge module and a cartridge module having an insufficient residual quantity.

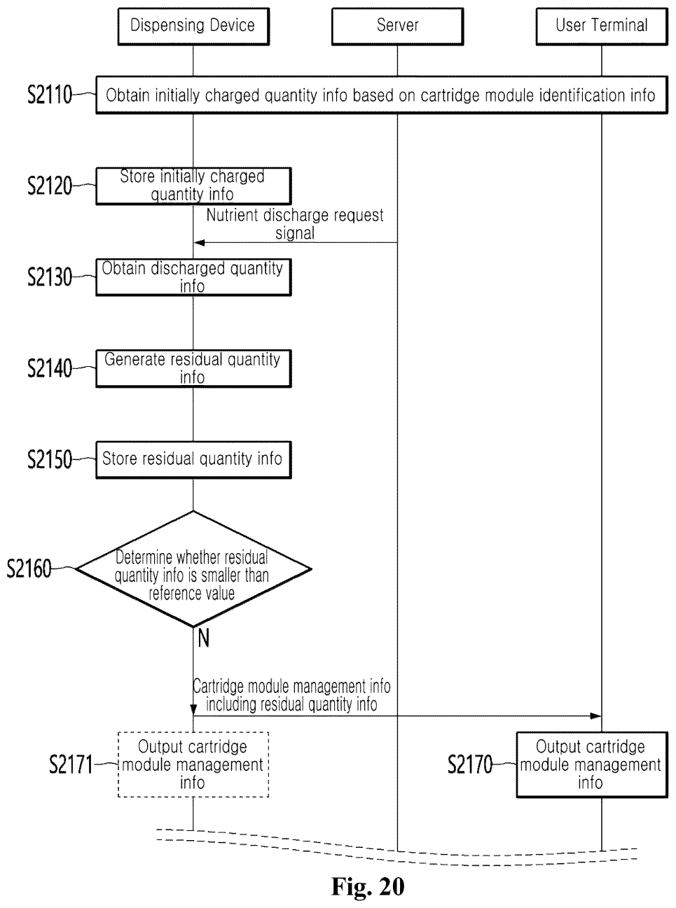

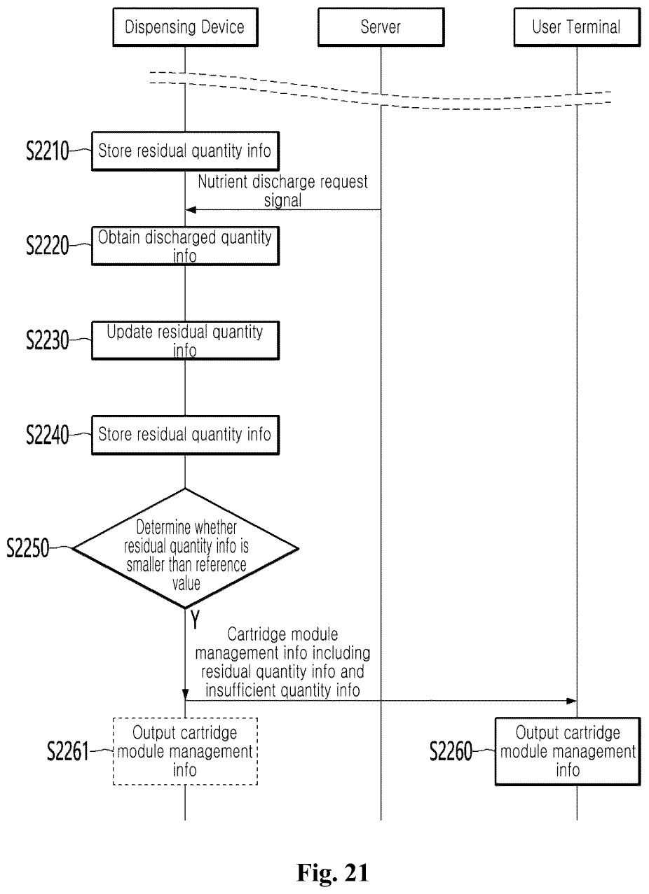

FIGS. 20 and 21 are flowcharts of a fourth embodiment in which the server, the user terminal, and the dispensing device perform a residual quantity management method while operating in conjunction with each other, wherein FIG. 20 is a flowchart of a case where a cartridge module is newly installed in the dispensing device and FIG. 21 is a flowchart of a case that is performed after a cartridge module has been used. Referring to FIGS. 20 and 21, at least one of the server, the user terminal, and the dispensing device may obtain initially charged quantity information based on cartridge identification information, the dispensing device may generate or update residual quantity information based on the discharged quantity information, and the information included in the cartridge module management information may vary depending on whether the residual quantity information is smaller than the reference value.

Referring to FIG. 20, at least one of the server, the user terminal, and the dispensing device may obtain initially charged quantity information based on cartridge identification information in step S2110. The content described in conjunction with FIG. 18 may be applied in the same manner, so that a redundant description is omitted.

The dispensing device may store the initially charged quantity information in step S2120.

The dispensing device may receive a nutrient discharge request signal from the server.

The dispensing device may discharge one or more nutrients in response to the nutrient discharge request signal.

As the nutrients are discharged, the dispensing device may obtain discharged quantity information in step S2130.

The dispensing device may generate residual quantity information based on the obtained discharged quantity information in step S2140. The dispensing device may store the residual quantity information in step S2150.

The dispensing device may determine whether the residual quantity information is smaller than the reference value in step S2160. When it is determined that the residual quantity information is not smaller than the reference value, the dispensing device may transmit cartridge module management information including the residual quantity information to the user terminal.

The user terminal may output the received cartridge module management information in step S2170. Accordingly, a user may determine the residual quantity of nutrients for each cartridge module.

In some cases, the dispensing device may output the cartridge module management information in step S2171. Accordingly, the user may determine the residual quantity of nutrients for each cartridge module.

Referring to FIG. 21, the dispensing device may store the residual quantity information of the used cartridge module in step S2210.

The dispensing device may receive a nutrient discharge request signal from the server.

The dispensing device may discharge one or more nutrients in response to the nutrient discharge request signal.

As the nutrients are discharged, the dispensing device may obtain discharged quantity information in step S2220.

The dispensing device may update the residual quantity information based on the discharged quantity information in step S2230. The dispensing device may store the updated residual quantity information in step S2240.

The dispensing device may determine whether the residual quantity information is smaller than the reference value in step S2250. When it is determined that the residual quantity information is smaller than the reference value, the dispensing device may transmit cartridge module management information, including the residual quantity information and insufficient quantity information, to the user terminal.

The user terminal may output the received cartridge module management information in step S2260. Accordingly, a user may determine the residual quantity of nutrients for each cartridge module and a cartridge module having an insufficient residual quantity.

In some cases, the dispensing device may output the cartridge module management information in step S2261. Accordingly, a user may determine the residual quantity of nutrients for each cartridge module and a cartridge module having an insufficient residual quantity.

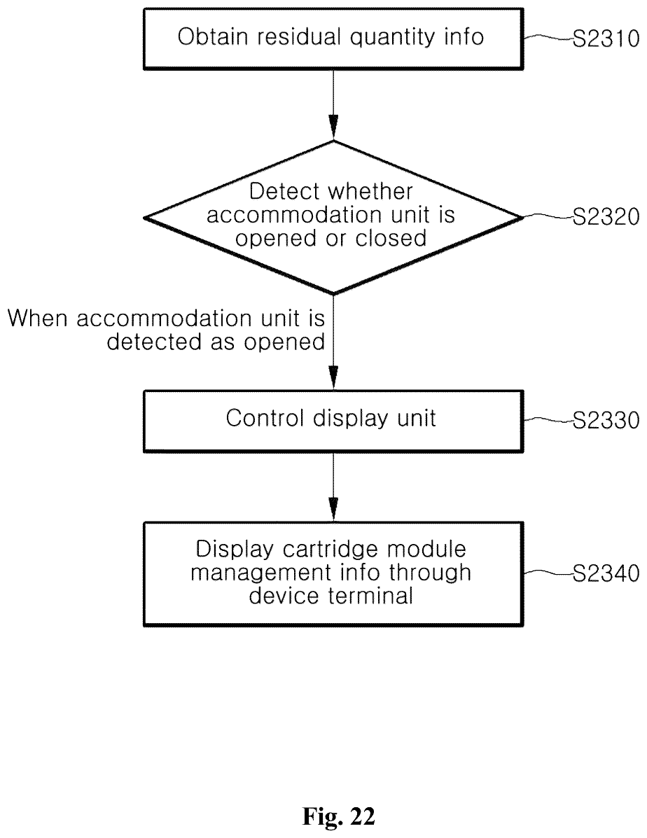

FIG. 22 is a flowchart of another example of a method of managing a cartridge module according to an embodiment, which is a flowchart of a method of operating the dispensing device according to the residual quantity of nutrients contained in a cartridge module.

In step S2310, the dispensing device may obtain residual quantity information. As an example, the residual quantity information may include information about the residual quantity of each of the plurality of cartridge modules mounted in the dispensing device.

In step S2320, the dispensing device may detect whether the accommodation unit is opened or closed. As an example, the dispensing device may detect whether the accommodation unit is opened or closed through the accommodation unit opening/closing detection unit.

In step S2330, when it is detected that the accommodation unit is opened, the dispensing device may control the display unit.

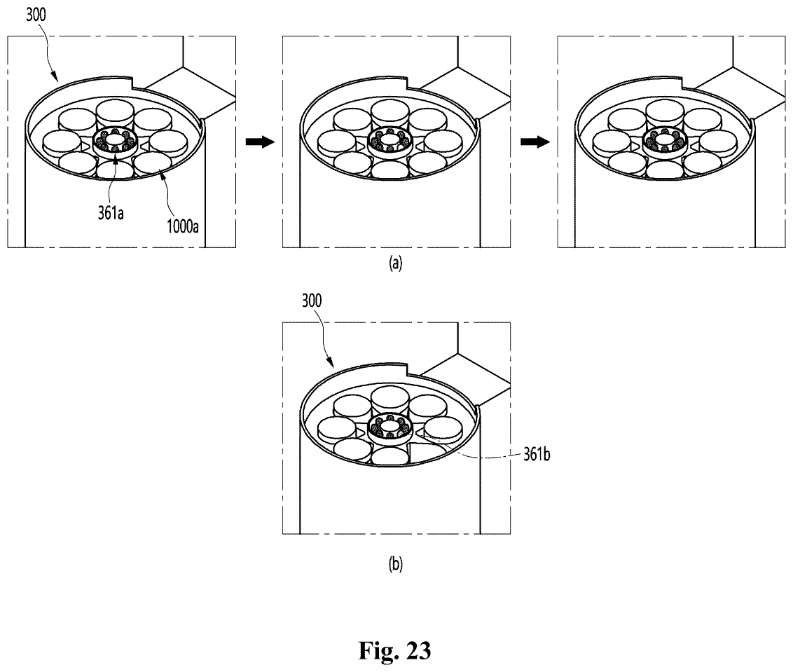

FIG. 23 is a diagram of an example in which the dispensing device 300 according to an embodiment controls the display unit.

The dispensing device may control the display unit based on the residual quantity of nutrients contained in the cartridge module. As an example, the dispensing device may display an indication lamp corresponding to a cartridge module whose residual quantity is smaller than a predetermined level and an indication lamp corresponding to a cartridge module whose residual quantity is equal to or larger than the predetermined level in different manners. As a more specific example, referring to drawing (a) of FIG. 23, the dispensing device 300 blinks an indication lamp 361a corresponding to a cartridge module 1000a whose residual quantity is smaller than the predetermined level, and turns on an indication lamp corresponding to a cartridge module whose residual quantity is equal to or larger than the predetermined level. As another more specific example, the dispensing device may display an indication lamp corresponding to an indication lamp corresponding to a cartridge module whose residual quantity is smaller than the predetermined level and an indication lamp corresponding to a cartridge module whose residual quantity is equal to or larger than the predetermined level in different colors.

The dispensing device may control the display unit based on whether cartridge modules have been mounted. An indication lamp may indicate whether a cartridge module has been mounted in a corresponding one of the cartridge insertion portions. As an example, the dispensing device may display an indication lamp corresponding to a cartridge insertion portion mounted with a cartridge module and an indication lamp corresponding to a cartridge insertion portion not mounted with a cartridge module in different manners. As a more specific example, referring to drawing (b) of FIG. 23, the dispensing device 300 may display an indication lamp corresponding to a cartridge insertion portion mounted with a cartridge module and an indication lamp 361b corresponding to a cartridge insertion portion not mounted with a cartridge module in different colors. As another more specific example, the dispensing device may turn on an indication lamp corresponding to a cartridge insertion portion in which a cartridge module has been mounted, and may blink an indication lamp corresponding to a cartridge insertion portion in which a cartridge module has not been mounted.

The dispensing device may control the display unit based on whether a cartridge module needs to be replaced. An indication lamp may indicate whether a corresponding cartridge module needs to be replaced. As an example, the dispensing device may display an indication lamp corresponding to a cartridge module that needs to be replaced differently from other indication lamps. As a more specific example, the dispensing device may blink an indication light corresponding to a cartridge module that needs to be replaced and turn on other indication lights. As another more specific example, the dispensing device may display an indication lamp corresponding to a cartridge module that needs to be replaced and other indication lamps in different colors.

The dispensing device may perform the operation of controlling the display unit when the accommodation unit is detected as opened. In this case, the dispensing device may not perform the operation of controlling the display unit when the accommodation unit is detected as closed. For example, the dispensing device may perform the operation of controlling the display unit based on whether the cartridge module has been mounted when the accommodation unit is detected as opened, and may not perform the operation when the accommodation unit is detected as closed. As another example, the dispensing device may perform the operation of controlling the display unit based on the residual quantity of nutrients contained in the cartridge module when the accommodation unit is detected as opened, and may not perform the operation when the accommodation unit is detected as closed. As still another example, the dispensing device may perform the operation of controlling the display unit based on whether the cartridge module needs to be replaced when the accommodation unit is detected as opened, and may not perform the operation when the accommodation unit is detected as closed.

Referring again to FIG. 22, in step S2340, the dispensing device may display cartridge module management information through the device terminal.

As an example, the dispensing device may display cartridge module management information when the accommodation unit is detected as opened, and may not display cartridge module management information when the accommodation unit is detected as closed.

As another example, the dispensing device may display cartridge module management information when the accommodation unit is detected as opened, and may display cartridge module management information for a predetermined period of time and then stop displaying the cartridge module management information when the accommodation unit is detected as closed.

Not all of the steps of FIG. 22 are essential, and the sequence of the steps may be changed or at least some of the steps may be omitted. As an example, step S2340 of displaying cartridge module management information through the device terminal may be performed before step S2330 of controlling the display unit. As another example, step S2340 of displaying cartridge module management information through the device terminal and step S2330 of controlling the display unit may be performed simultaneously. As still another example, one of step S2340 of displaying cartridge module management information through the device terminal and step S2330 of controlling the display unit may not be performed.

FIG. 24 is a flowchart of a more specific illustration of another example of the method of managing a cartridge module shown in FIG. 22. Not all of the steps of FIG. 24 are essential, and the sequence of the steps may be changed or at least some of the steps may be omitted.

In step S2410, the dispensing device may obtain residual quantity information. As for this, the content described in conjunction with step S2310 may be applied in the same manner, so that a redundant description is omitted.

In step S2420, the dispensing device may detect whether the accommodation unit is opened or closed. As for this, the content described in conjunction with step S2320 may be applied in the same manner, so that a redundant description is omitted.

When the accommodation unit is detected as closed, the dispensing device may detect whether the accommodation unit is opened or closed until the accommodation unit is detected as opened. As an example, the dispensing device may detect whether the accommodation unit is opened or closed again when a predetermined time has elapsed after the accommodation unit has been as closed. As another example, the dispensing device may detect whether the accommodation unit is opened or closed at predetermined intervals.

In step S2430, when the accommodation unit is detected as opened, the dispensing device may control the display unit. As an example, the dispensing device may control the display so that a cartridge module whose residual quantity is smaller than a predetermined level or a cartridge module that needs to be replaced can be identified. As for step S2430, the content described in conjunction with step S2330 may be applied in the same manner, so that a redundant description is omitted.

In step S2440, the dispensing device may detect whether a new cartridge module is installed. As an example, the dispensing device may detect whether a new cartridge module is installed through the cartridge module interface.

In step S2450, when a new cartridge module is detected as not mounted, the dispensing device may detect whether the accommodation unit is opened or closed. When the accommodation unit is detected as opened, the dispensing device may again detect whether a new cartridge module is installed. When the accommodation unit is detected as closed, the dispensing device may display cartridge module management information through the device terminal in step S2490. As an example, when the accommodation unit is detected as closed, the dispensing device may display cartridge module management information for a predetermined period of time.

In step S2460, when a new cartridge module is detected as mounted, the dispensing device may obtain the initially charged quantity information of the new cartridge module. As an example, the dispensing device may obtain initially charged quantity information from the memory chip of the new cartridge module.

In step S2470, the dispensing device may control the display unit. As for this, the content described in conjunction with step S2330 may be applied in the same manner, so that a redundant description is omitted.

In step S2480, the dispensing device may detect whether the accommodation unit is opened or closed. As for this, the content described in conjunction with step S2320 may be applied in the same manner, so that a redundant description is omitted.

When the accommodation unit is detected as opened, the dispensing device may detect whether the accommodation unit is opened or closed until the accommodation unit is detected as closed. As an example, the dispensing device may detect whether the accommodation unit is opened or closed again when a predetermined time has elapsed after the accommodation unit has been detected as opened. As another example, the dispensing device may detect whether the accommodation unit is opened or closed at predetermined intervals.

In step S2490, when the accommodation unit is detected as closed, the dispensing device may display cartridge module management information through the device terminal. As an example, when the accommodation unit is detected as closed, the dispensing device may display cartridge module management information for a predetermined period of time.

The method according to the embodiment may be performed by processing logic including hardware, firmware, software, or a combination thereof. The method according to the embodiment may be performed by a processor configured to execute code stored in a non-transitory computer-readable medium. Examples of the non-transitory computer-readable medium include magnetic media such as a hard disk, a floppy disk, and a magnetic tape, optical media such as CD-ROM and a DVD, magneto-optical media such as a floptical disk, and hardware devices specifically configured to store and perform program instructions such as ROM, RAM, flash memory, etc.

A non-transitory computer-readable medium may store one or more instructions. The one or more instructions may cause the dispensing device or the server to perform the method according to an embodiment when executed by one or more processors of the dispensing device or server.

Although the present application has been described based on the embodiments in the foregoing description, the present application is not limited thereto. It will be apparent to those having ordinary skill in the art that various changes or modifications may be made within the spirit and scope of the present application. Therefore, it is noted that such changes or modifications fall within the scope of the attached patent claims.

### Implementation Form of the Invention

[symbol]

1. A dispensing device, comprising:
an accommodation unit configured to provide an internal space for accommodating a plurality of cartridge modules;
a discharge unit configured to discharge tablets from each of the plurality of cartridge modules;
detection sensors provided to corresponding to the plurality of cartridge module, respectively, and each configured to count a number of tablets discharged from each of the plurality of cartridge modules; and
a control unit electrically connected to electrodes of the accommodation unit, the discharge unit, and the detection sensors, and configured to control discharge of the tablets;
wherein the control unit:
performs control to discharge the tablets from at least one of the plurality of cartridge modules through the discharge unit in response to a discharge request signal for the tablets; and
after the tablets have been discharged from the at least one cartridge module, performs control to update residual quantity information for the at least one cartridge module based on discharged quantity information about the number of tablets detected by the detection sensor.

2. The dispensing device of claim 1, wherein:
each of the plurality of cartridge modules includes a cartridge identification unit configured to store information about the cartridge module;
the accommodation unit has cartridge module interfaces each electrically connected to the cartridge identification unit; and
the control unit performs control to obtain information about the cartridge module from the cartridge identification unit through the cartridge module interface when each of the plurality of cartridge modules is mounted in the accommodation unit.

3. The dispensing device of claim 2, wherein:
the cartridge identification unit is provided in a form of a read/write-enabled memory chip;
the information about the cartridge module includes the residual quantity information; and
the control unit:
performs control to obtain the residual quantity information from the memory chip when each of the plurality of cartridge modules is mounted in the accommodation unit; and
after the residual quantity information has been updated, performs control to store the residual quantity information in the memory chip.

4. The dispensing device of claim 1, further comprising an output unit configured to display information about the plurality of cartridge modules;
wherein the output unit:
displays a plurality of images for the plurality of cartridge modules to correspond to an arrangement of the plurality of cartridge modules in the accommodation unit; and
displays information about tablets accommodated in each of the plurality of cartridge modules and residual quantity information together in each of the plurality of images.

5. The dispensing device of claim 4, wherein the output unit further displays insufficient quantity information for an image representative of a cartridge module whose residual quantity information is smaller than a reference value among the plurality of images.

6. The dispensing device of claim 1, wherein:
the accommodation unit comprises:
a top cover configured to cover the inner space of the accommodation unit; and
a display unit including a plurality of indication lamps located in respective portions corresponding to the plurality of cartridge modules in the internal space of the accommodation unit; and
the control unit:
when the accommodation unit is opened, performs control to blink a target indication lamp corresponding to a cartridge module whose residual quantity is smaller than a predetermined reference value among the plurality of indicator lamps; and
when the accommodation unit is closed or the cartridge module whose residual quantity is smaller than the predetermined reference value is separated from the accommodation unit, performs control to stop blinking the target indication lamp.

7. The dispensing device of claim 6, wherein the control unit, when a new cartridge module is mounted after the cartridge module whose residual quantity is smaller than the predetermined reference value has been separated, controls the target indication lamp by using at least one of identification information and residual quantity information of the new cartridge module.

8. The dispensing device of claim 7, wherein the control unit performs control to obtain the at least one of the identification information and residual quantity information of the new cartridge module from a memory chip included in the new cartridge module.

9. The dispensing device of claim 6, wherein the control unit, when the cartridge module whose residual quantity is smaller than the predetermined reference value is separated and then a cartridge module whose residual quantity is smaller than the predetermined reference value is mounted, blinks again the target indication lamp whose blinking was stopped.

10. A user terminal, comprising:
a terminal storage unit including memory;
a terminal control unit including a processor electrically connected to the terminal storage unit, and configured to execute commands stored in the terminal storage unit;
a terminal communication unit configured to transmit and receive information to and from a server or dispensing device under control of the terminal control unit; and
a terminal output unit configured to output results according to operation of the terminal control unit;
wherein the terminal control unit:
performs control to receive residual quantity information about a plurality of cartridge modules mounted in the dispensing device from the server or dispensing device and to output the residual quantity information through the terminal output unit; and
after one or more tablets have been discharged from the plurality of cartridge modules, performs control to update and output the residual quantity information.

11. The user terminal of claim 10, wherein the terminal output unit displays the residual quantity information together with information about tablets accommodated in each of the plurality of cartridge modules.

12. The user terminal of claim 10, wherein the terminal output unit displays a plurality of images for the plurality of cartridge modules to correspond to an arrangement of the plurality of cartridge modules in the dispensing device.

13. The user terminal of claim 10, wherein:
the terminal control unit, when the residual quantity information for at least one of the plurality of cartridge modules is smaller than a preset reference value, performs control to receive insufficient quantity information for the at least one module from the server or dispensing device; and
the terminal output unit outputs the insufficient quantity information for the at least one cartridge module together with the residual quantity information.

14. The user terminal of claim 10, wherein the terminal output unit displays, among the plurality of images representative of the plurality of cartridge modules, an image representative of a cartridge module whose residual quantity information is smaller than a predetermined reference value so that the image can be distinguished from an image representative of a cartridge module whose residual quantity information is equal to or larger than the reference value.
